SEQUENCE LISTING (12 sequences)

SEQ ID NO: 1 - protein, 35 aa, Homo sapiens
DLHPHKHHSHEQHPHGHHPHAHHPHEHDTHRQHPH

SEQ ID NO: 2 - protein, 507 aa, Homo sapiens
VSPTDCSAVEPEAEKALDLINKRRRDGYLFQLLRIADAHLDRVENTTVYYLVLDVQESDCSVLSRKYWNDCEPPDSRRPSEIVIGQCKVIATRHSHESQDLRVIDFNCTTSSVSSALANTKDSPVLIDFFEDTERYRKQANKALEKYKEENDDFASFRVDRIERVARVRGGEGTGYFVDFSVRNCPRHHFPRHPNVFGFCRADLFYDVEALDLESPKNLVINCEVFDPQEHENINGVPPHLGHPFHWGGHERSSTTKPPFKPHGSRDHHHPHKPHEHGPPPPPDERDHSHGPPLPQGPPPLLPMSCSSCQHATFGTNGAQRHSHNNNSSDLHPHKHHSHEQHPHGHHPHAHHPHEHDTHRQHPHGHHPHGHHPHGHHPHGHHPHGHHPHCHDFQDYGPCDPPPHNQGHCCHGHGPPPGHLRRRGPGKGPRPFHCRQIGSVYRLPPLRKGEVLPLPEANFPSFPLPHHKHPLKPDNQPFPQSVSESCPGKFKSGFPQVSMFFTHTFPK

SEQ ID NO: 3 - protein, 21 aa, unidentified
MWWRLWWLLLLLLLLWPMVWA

SEQ ID NO: 4 - DNA, 8061 bp, unidentified
  source 1..8061: mol_type = other DNA
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggctgcaggtcgacggatccagcccgggccccccgcgggcgcgcgcgcgcgcaaaaaaggcgggcggcggtccgggcggcgtgcgcgcgcgcggcgggcgtggggggcggggccgcgggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggccagacgccgaaaacgggccccccccaaaacacaccccccgggggtcgcgcgcggccctttaaagcggtggcggcgggcagcccgggccccccgcggccgagactagcgagttagacaggcaagcactactcgcctctgcacgcacatgcttgcctgtcaaactctaccaccccggcacgctctctgtctccatggcccgccgccgccgccatcgcggcccccgccgcccccggccgcccgggcccacgggcgccgtcccaaccgcacagtcccaggtaaccctcgaccctagatgcgccgacccggaaacgccatataaggagcaggaaggatcccccgccggaacagaccttatttgggcagcgccttatatggagtggcccaatatggccctgccgcttccggctctgggaggagggcgagcgggggttggggcgggggcaactgggaactccaggcgcctggcccgggaggccactgctgctgttccaatactaggctttccaggacctgagcgctcgcgatgccggagcgggtcgcagggtggagtgcccaccactcttggatgggagggcttcacgtcctccgggtcctcccggccggtccttccatattagggcttcctgcttcccatatatggccatgtacgtcacgcggaagcgggcccgtgctgttccagacccttgaaatagaggccgattcggggagtcgcgagagatcccagcgcgcagaacttggggagccgccgccgcgattcgccgccgccgccagcttccggtcgagtcgaggtcgaggtcgacggtatcgataagcttgggctgcaggtcgatcgactctaggtttatctgcagaattcgcccttaaaataggccttaattaaagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttatgaaggcactcattgcagcactgcttttgatcacattgcagtattcgtgtgccgtgagtcccactgactgcagtgctgttgagccggaggctgagaaagctctagacctgatcaataaaaggcgacgggatggctaccttttccaattgctgcggattgctgatgcccacttggacagagtggaaaatacaactgtatattacttagtcttagatgtgcaagaatcggactgttcggtcctatccaggaaatactggaatgactgtgagccacctgattccagacgtccatctgaaatagtgatcggacaatgtaaggtaatagctacaagacattcccatgaatctcaggacctcagagtgattgactttaactgcaccacaagttctgtctcttcagcactggccaataccaaagatagtccggtcctcatagatttctttgaggatactgagcgctacagaaaacaagccaacaaagcccttgagaagtacaaagaggagaatgatgactttgcctctttcagagtggaccgaatcgagagagttgcaagagtgagaggaggggaaggaactggttacttcgtggacttctctgtgcggaactgccccagacaccatttccccagacaccccaatgtctttggattctgcagagcagatttgttctatgatgtagaagccttggacttggaaagcccgaaaaaccttgtcataaactgtgaagtcttcgaccctcaggaacatgagaacatcaatggtgtaccgcctcatttgggacatcccttccactggggtgggcatgagcgttcttctaccaccaagcctccattcaagccccatggatctagagatcatcatcatccccacaagccacacgaacatggacccccacctcctccagatgaaagagatcactcacatggacccccacttccacaaggccctcctccactattgcccatgtcctgctcaagttgtcaacatgccacttttggcacaaatggggcccaaagacattctcataataataattccagtgacctccatccccataagcatcattcccatgaacagcatccccacggacaccatccccatgcacaccatcctcatgaacatgatacccatagacagcatccccatggacaccacccccatggacaccatcctcatggacaccacccccatggacaccatccccatggacaccatccccactgccatgatttccaagactatggaccttgtgacccaccaccccataaccaaggtcactgttgccatggccacggcccaccacctgggcacttaagaaggcgaggcccaggtaaaggaccccgtcccttccattgcagacaaattggatctgtgtaccgactccctcctctaagaaaaggtgaggtgctgccacttcctgaggccaattttcccagcttcccattgccgcaccacaaacatcctctaaagccagacaatcagccctttcctcaatcagtctctgaatcatgtccagggaagttcaagagtgggtttccacaagtttccatgttttttacacatacatttccaaaataaggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaagataaactagagtcgacctgcagatctctagatccggagagacgatggcaggagccgcgcatatatacgcttggagccagcccgccctcacagggcgggccgcctcgggggcgggactggccaatcggcggccgccagcgcggcggggcccggccaaccagcgtccgccgagtcttcggggcccggcccattgggcgggagttaccgcccaatgggccgggccgcccacttcccggtatggtaattaaaaacttgcaagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttgggcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtataagttagccctggcccgacagtctggtcgcatttgcacctcggcactcggagcgagacgcagcagccaggcagactcgggccgccccctctccgcatcaccacagaagccccgcctacgttgcgacccccagggaccctccgtccgcgaccctccagccgcatacgacccccatggagccccgccccggagcgagtacccgccggcctgagggccgcccccagcgcgaggtgaggggccgggcgccatgtctggggcgccatattggggggcgccatattggggggcgccatgttgggggacccccgacccttacactggaaccggccgccatgttgggggacccccactcatacacgggagccgggcgccatgttggggcgccatgttagggggcgtggaaccccgtgacactatatatacagggaccgggggcgccatgttagggggtgcggaaccccctgaccctatatatacagggaccggggtcgccctgttgggggtcgccatgtgaccccctgactttatatatacagacccccaacacatacacatggcccctttgactcagacgcagggcccggggtcgccgtgggaccccctgactcatacacagagacacgcccccacaacaaacacacaaggaccggggtcgccgtgttgggggcgtggtccccactgactcatacgcaggccccccttactcacacgcatctaggggggtggggaggagccgcccgccatatttgggggacgccgtgggacccccgactccggtgcgtctggagggcgggagaagagggaagaagaggggtcgggatccccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 5 - DNA, 535 bp, unidentified
  source 1..535: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgct

SEQ ID NO: 6 - DNA, 54 bp, unidentified
  source 1..54: mol_type = other DNA
atgaaggcactcattgcagcactgcttttgatcacattgcagtattcgtgtgcc

SEQ ID NO: 7 - DNA, 1524 bp, unidentified
  source 1..1524: mol_type = other DNA
gtgagtcccactgactgcagtgctgttgagccggaggctgagaaagctctagacctgatcaataaaaggcgacgggatggctaccttttccaattgctgcggattgctgatgcccacttggacagagtggaaaatacaactgtatattacttagtcttagatgtgcaagaatcggactgttcggtcctatccaggaaatactggaatgactgtgagccacctgattccagacgtccatctgaaatagtgatcggacaatgtaaggtaatagctacaagacattcccatgaatctcaggacctcagagtgattgactttaactgcaccacaagttctgtctcttcagcactggccaataccaaagatagtccggtcctcatagatttctttgaggatactgagcgctacagaaaacaagccaacaaagcccttgagaagtacaaagaggagaatgatgactttgcctctttcagagtggaccgaatcgagagagttgcaagagtgagaggaggggaaggaactggttacttcgtggacttctctgtgcggaactgccccagacaccatttccccagacaccccaatgtctttggattctgcagagcagatttgttctatgatgtagaagccttggacttggaaagcccgaaaaaccttgtcataaactgtgaagtcttcgaccctcaggaacatgagaacatcaatggtgtaccgcctcatttgggacatcccttccactggggtgggcatgagcgttcttctaccaccaagcctccattcaagccccatggatctagagatcatcatcatccccacaagccacacgaacatggacccccacctcctccagatgaaagagatcactcacatggacccccacttccacaaggccctcctccactattgcccatgtcctgctcaagttgtcaacatgccacttttggcacaaatggggcccaaagacattctcataataataattccagtgacctccatccccataagcatcattcccatgaacagcatccccacggacaccatccccatgcacaccatcctcatgaacatgatacccatagacagcatccccatggacaccacccccatggacaccatcctcatggacaccacccccatggacaccatccccatggacaccatccccactgccatgatttccaagactatggaccttgtgacccaccaccccataaccaaggtcactgttgccatggccacggcccaccacctgggcacttaagaaggcgaggcccaggtaaaggaccccgtcccttccattgcagacaaattggatctgtgtaccgactccctcctctaagaaaaggtgaggtgctgccacttcctgaggccaattttcccagcttcccattgccgcaccacaaacatcctctaaagccagacaatcagccctttcctcaatcagtctctgaatcatgtccagggaagttcaagagtgggtttccacaagtttccatgttttttacacatacatttccaaaataa

SEQ ID NO: 8 - DNA, 225 bp, unidentified
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 9 - DNA, 8063 bp, unidentified
  source 1..8063: mol_type = other DNA
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggctgcaggtcgacggatccagcccgggccccccgcgggcgcgcgcgcgcgcaaaaaaggcgggcggcggtccgggcggcgtgcgcgcgcgcggcgggcgtggggggcggggccgcgggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggccagacgccgaaaacgggccccccccaaaacacaccccccgggggtcgcgcgcggccctttaaagcggtggcggcgggcagcccgggccccccgcggccgagactagcgagttagacaggcaagcactactcgcctctgcacgcacatgcttgcctgtcaaactctaccaccccggcacgctctctgtctccatggcccgccgccgccgccatcgcggcccccgccgcccccggccgcccgggcccacgggcgccgtcccaaccgcacagtcccaggtaaccctcgaccctagatgcgccgacccggaaacgccatataaggagcaggaaggatcccccgccggaacagaccttatttgggcagcgccttatatggagtggcccaatatggccctgccgcttccggctctgggaggagggcgagcgggggttggggcgggggcaactgggaactccaggcgcctggcccgggaggccactgctgctgttccaatactaggctttccaggacctgagcgctcgcgatgccggagcgggtcgcagggtggagtgcccaccactcttggatgggagggcttcacgtcctccgggtcctcccggccggtccttccatattagggcttcctgcttcccatatatggccatgtacgtcacgcggaagcgggcccgtgctgttccagacccttgaaatagaggccgattcggggagtcgcgagagatcccagcgcgcagaacttggggagccgccgccgcgattcgccgccgccgccagcttccggtcgagtcgaggtcgaggtcgacggtatcgataagcttgggctgcaggtcgatcgactctaggtttatctgcagaattcgcccttaaaataggccttaattaaagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccaccatgtggtggagactgtggtggctgctgctgctgctgctgctgctgtggcccatggtgtgggccgccgtgagtcccactgactgcagtgctgttgagccggaggctgagaaagctctagacctgatcaataaaaggcgacgggatggctaccttttccaattgctgcggattgctgatgcccacttggacagagtggaaaatacaactgtatattacttagtcttagatgtgcaagaatcggactgttcggtcctatccaggaaatactggaatgactgtgagccacctgattccagacgtccatctgaaatagtgatcggacaatgtaaggtaatagctacaagacattcccatgaatctcaggacctcagagtgattgactttaactgcaccacaagttctgtctcttcagcactggccaataccaaagatagtccggtcctcatagatttctttgaggatactgagcgctacagaaaacaagccaacaaagcccttgagaagtacaaagaggagaatgatgactttgcctctttcagagtggaccgaatcgagagagttgcaagagtgagaggaggggaaggaactggttacttcgtggacttctctgtgcggaactgccccagacaccatttccccagacaccccaatgtctttggattctgcagagcagatttgttctatgatgtagaagccttggacttggaaagcccgaaaaaccttgtcataaactgtgaagtcttcgaccctcaggaacatgagaacatcaatggtgtaccgcctcatttgggacatcccttccactggggtgggcatgagcgttcttctaccaccaagcctccattcaagccccatggatctagagatcatcatcatccccacaagccacacgaacatggacccccacctcctccagatgaaagagatcactcacatggacccccacttccacaaggccctcctccactattgcccatgtcctgctcaagttgtcaacatgccacttttggcacaaatggggcccaaagacattctcataataataattccagtgacctccatccccataagcatcattcccatgaacagcatccccacggacaccatccccatgcacaccatcctcatgaacatgatacccatagacagcatccccatggacaccacccccatggacaccatcctcatggacaccacccccatggacaccatccccatggacaccatccccactgccatgatttccaagactatggaccttgtgacccaccaccccataaccaaggtcactgttgccatggccacggcccaccacctgggcacttaagaaggcgaggcccaggtaaaggaccccgtcccttccattgcagacaaattggatctgtgtaccgactccctcctctaagaaaaggtgaggtgctgccacttcctgaggccaattttcccagcttcccattgccgcaccacaaacatcctctaaagccagacaatcagccctttcctcaatcagtctctgaatcatgtccagggaagttcaagagtgggtttccacaagtttccatgttttttacacatacatttccaaaataaggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaagataaactagagtcgacctgcagatctctagatccggagagacgatggcaggagccgcgcatatatacgcttggagccagcccgccctcacagggcgggccgcctcgggggcgggactggccaatcggcggccgccagcgcggcggggcccggccaaccagcgtccgccgagtcttcggggcccggcccattgggcgggagttaccgcccaatgggccgggccgcccacttcccggtatggtaattaaaaacttgcaagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttgggcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtataagttagccctggcccgacagtctggtcgcatttgcacctcggcactcggagcgagacgcagcagccaggcagactcgggccgccccctctccgcatcaccacagaagccccgcctacgttgcgacccccagggaccctccgtccgcgaccctccagccgcatacgacccccatggagccccgccccggagcgagtacccgccggcctgagggccgcccccagcgcgaggtgaggggccgggcgccatgtctggggcgccatattggggggcgccatattggggggcgccatgttgggggacccccgacccttacactggaaccggccgccatgttgggggacccccactcatacacgggagccgggcgccatgttggggcgccatgttagggggcgtggaaccccgtgacactatatatacagggaccgggggcgccatgttagggggtgcggaaccccctgaccctatatatacagggaccggggtcgccctgttgggggtcgccatgtgaccccctgactttatatatacagacccccaacacatacacatggcccctttgactcagacgcagggcccggggtcgccgtgggaccccctgactcatacacagagacacgcccccacaacaaacacacaaggaccggggtcgccgtgttgggggcgtggtccccactgactcatacgcaggccccccttactcacacgcatctaggggggtggggaggagccgcccgccatatttgggggacgccgtgggacccccgactccggtgcgtctggagggcgggagaagagggaagaagaggggtcgggatccccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 10 - skipped

SEQ ID NO: 11 - DNA, 66 bp, unidentified
  source 1..66: mol_type = other DNA
atgtggtggagactgtggtggctgctgctgctgctgctgctgctgtggcccatggtgtgggccgcc

SEQ ID NO: 12 - DNA, 11765 bp, unidentified
  source 1..11765: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttggagacgtcctcctcgtcccccacgaaaacaagctcggtttccagccagcggttaaggatgctgagggagcgcaggtggttaatgtcgggacactgggaggtgtagaagtacctctcggggtcggggcactttggaatctggatcgccaggtccgccggcgcgctctggcccgtatgggccgtcagagcgggggagagggctggggccgcggaatccatggcagcgggggagcgcgtgggacggcgacgacagtggcggcgggcctggcgcggagggggtttgtcggtcacagcgcgcagctcgtgcagacaatgttgtcgtcgccgccaaagaccccgctgttggtcgccttgcgaaccttgcagtagtacatccctgtttttagtccgcgcttatatgcgtggaccagaaggcggaccagggtggaggctgggagggtcccgtccgccttctccgtgacatacagggtcatggattggctatggtcgacgtagggggcgcggtccgcacacaggtcgatcagcaacttctggtcgtagtcaaacgcggtcttgaatcgccggagggggtgggtgggctccaggcacgggagcgcctgcgccacggaccactgcttggcgtcgagactgtccatcacctccaggaggcgcttcccgctaaacgtgcgttccagttcctttagcaggagcgtgttggggcgcagcgtctcgccgtcccgggtcaccttgctaaacaggttggtgaacaggggggcaaagccctcgctgacgtccgagatcttattaattaaagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccaccatgtggtggagactgtggtggctgctgctgctgctgctgctgctgtggcccatggtgtgggccgccgtgagtcccactgactgcagtgctgttgagccggaggctgagaaagctctagacctgatcaataaaaggcgacgggatggctaccttttccaattgctgcggattgctgatgcccacttggacagagtggaaaatacaactgtatattacttagtcttagatgtgcaagaatcggactgttcggtcctatccaggaaatactggaatgactgtgagccacctgattccagacgtccatctgaaatagtgatcggacaatgtaaggtaatagctacaagacattcccatgaatctcaggacctcagagtgattgactttaactgcaccacaagttctgtctcttcagcactggccaataccaaagatagtccggtcctcatagatttctttgaggatactgagcgctacagaaaacaagccaacaaagcccttgagaagtacaaagaggagaatgatgactttgcctctttcagagtggaccgaatcgagagagttgcaagagtgagaggaggggaaggaactggttacttcgtggacttctctgtgcggaactgccccagacaccatttccccagacaccccaatgtctttggattctgcagagcagatttgttctatgatgtagaagccttggacttggaaagcccgaaaaaccttgtcataaactgtgaagtcttcgaccctcaggaacatgagaacatcaatggtgtaccgcctcatttgggacatcccttccactggggtgggcatgagcgttcttctaccaccaagcctccattcaagccccatggatctagagatcatcatcatccccacaagccacacgaacatggacccccacctcctccagatgaaagagatcactcacatggacccccacttccacaaggccctcctccactattgcccatgtcctgctcaagttgtcaacatgccacttttggcacaaatggggcccaaagacattctcataataataattccagtgacctccatccccataagcatcattcccatgaacagcatccccacggacaccatccccatgcacaccatcctcatgaacatgatacccatagacagcatccccatggacaccacccccatggacaccatcctcatggacaccacccccatggacaccatccccatggacaccatccccactgccatgatttccaagactatggaccttgtgacccaccaccccataaccaaggtcactgttgccatggccacggcccaccacctgggcacttaagaaggcgaggcccaggtaaaggaccccgtcccttccattgcagacaaattggatctgtgtaccgactccctcctctaagaaaaggtgaggtgctgccacttcctgaggccaattttcccagcttcccattgccgcaccacaaacatcctctaaagccagacaatcagccctttcctcaatcagtctctgaatcatgtccagggaagttcaagagtgggtttccacaagtttccatgttttttacacatacatttccaaaataaggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaataaccggtagacgtccccgccgagcgacgtggggttcccccgaatgcaacgacggcggtcccgccgtcgctgccggctccgatgtttgtcaccgcaacgaagggagcgggggatgccgcggcccccgggtcctgggggcgcccgcgcaccacgtctccgtcgataatcatggtgcagttggaaccacattggacaaagttgctatcgctgatgcggtaggacgcggacccgggcgtcttgttggaaagcaccatcacgccattgactcgctggcagtacacgtggtggccgtgggcgagaggggccccggcggcctccccctgggtggctgcgctggggccgccggcctcctgtcccccaaccggggcccgcgcttcgacgggagaggatgcggttgggcggctggccatttcaacagacgcggcgggtttcggcgggctccagaggggacgtcggcgggtccggcgagtggtggggtcgagattcgacaaggccgctgcctgacgaatcgcaccgacagccaaagaagataggcgagagcaggtccggaaggcgggagcgcaaaggaaaccggggagggacgcaagctcacaggagcacggcgaccgacaagtttccaaagcacccacctgtggtaccctaaaccacccgcgctggctttttatccacatcgcgcaggggtggggcgcgggccagtttcagtgaaacccaaaaggacacgccgatgtcctgagtcacgggggtgtgttccttccatgtatcccatttgcattttgtggcttcctcccgcccaaaggacgttacaacactcgcgtttcgggtttcagtggctttatttggcggacgtccgggtcaacgaaccacgagtgacagcggggaaggggttgggggcggagcgttgtgtgggcggggcgtttgctacgctcacgcgcatgcccgccactcgccggggcgccacaccacgccttccagaataacaacggggacgctgaccgccccaaagcgctcggtgcggtgcaggcagcggggactatcctcgggcatcatgccgagctctgcaaaggcggcgtacgtacaggtgcgtgcggtggggcgcccccgaaggtccggctgccagcggtacagcctgtcggcgaactgggggttcgtgacctggccagccgagcaacgcggggtttggggattggcggccaggcccgcgagggggaagtcggggtttcggtttggggccgggggcgtcatgtcctcggtgccgtgaatcgtgttggtgatccggaggcgcccaaacagccgctccagggccggctccaacccgcgcggggggagctggctcttgatcacgtacacacaacacagctcctggtcccggcgctggcggtaagtgaataccaggtacagaaacgccgcgcccgggccccccagccccagctcggcgtccaggtccacgaacgcacacgccgggaggatcacggccgagcggggccggcccgggtggccggtgtgcgccgcctcctggggcccggcgcacacggcggtgacgctcgctagctcgtcctgggtgacccacgacaccagccccccgaaaaaccgcatgacctcgtgggggaagacgtgcgtgtgaaccatggcgcgcagacactcggaaaaacgatccaggcgcgcccccacccgcgtcccgcggtagttggccgtgacgtgggcccgtaccgcatcggcggcgtcgcgcgcgtcgtacgaggcggcacacgccgccaccaggaagttaaacgacacgaggcccgcgcgcgtgcacccgctctcggctcgccccgaccccagggcggtcgcctccatcagctggccccaggcctcgcccagccgctcggtctgccggcctggcggggcgcgctggtgggccaggtgaggcaggtcggcggggtgccgcagcgccagaagcagcgtcccggcacgatccgcgttgggttggcacagatccgtcaggatcacttggcgggttagatgatggcgcggggagccgatcagggccgcccccccgcgcatggtgccccgcaggatcttgtccagggcctgttcggtatcgtcgttgggggtcagcgccccagggggcgcgtctgtgccgtccaggccaagcaaccacagcgtgctggccgccctccggggtcccgaccccctggggagcactgggccgtcgcggcgagagatcggggggcgcaggacgcaccgaagcaggccctgccccgcggtatcgcgtgtggtgggggggagttccacggtactcccgccccacacggaaggggttgcgggtagcggattggtcttcattgcgaccacagatcgcgacctagcaagacccgggggaggggggggtcgaaaggacactcacgcaaggcggaaccacccaccccacagccaaacgcggctaccgcctccggtgggttgtgttggccgactggcccggcgcgtccgtatacacacgtttaaagggccatttgggccgccccgcagaaagtccgcaacccacgccgtccgggctgcccgcacatccggacaatcccccgggcctgggtccgcgaacgggatgccgggacttaagtggccgtataacaccccgcgaagacgcggggtactcgcaacgcctgcggggttcctggagggccgcaggggatcgataattcgccgctccctacagcgcacgacagtcattcccgcccggtctcgtcgttggtctacgctgtcccccacccacgcgagccgggcgtcatggcagaccgcggtctcccgtccgaggcccccgtcgtcacgacctcacccgccggtccgccctcggacggacctatgcagcgcctattggcgagcctagccggccttcgccaaccgccaacccccacggccgagacggcaaacggggcggacgacccggcgtttctggccacggccaagctgcgcgccgccatggcggcgtttctgttgtcgggaacggccatcgccccggcagacgcgcgggcatgctggcggccgctgctggaacacctgtgcgcgctccaccgggcccacgggcttccggagacggcgctcttggccgagaacctccccgggttgctcgtacaccgcttggtggtggctctccccgaggcccccgaccaggccttccgggagatggaggtcatcaaggacaccatcctcgcggtcaccggctccgacacgtcccatgcgctggattccgccggcctgcgcaccgcggcggccctggggccggtccgcgtccgccagtgcgccgtggagtggatagaccgctggcaaaccgtcaccaagagctgcttggccatgagcccgcggacctccatcgaggcccttggggagacgtcgctcaagatggcgccggtcccgttggggcagcccagcgcgaaccttaccaccccggcgtacagcctgctcttccccgccccgttcgtgcaagagggcctccggttcttggccctggtgagtaatcgggtgacgctgttctcggcgcacctccagcgcatagacgacgcgaccctcactcccctcacacgggccctctttacgttggccctggtggacgagtacctgacgacccccgagcggggggctgtggtcccgccgcccctgttggcgcagtttcagcacaccgtgcgggagatcgacccggccataatgattccgccactggaggccaacaagatggttcgcagccgcgaggaggtgcgcgtgtcgacggccctcagccgcgtcagcccgcgctcggcctgtgcgcccccggggacgctaatggcgcgcgtgcggacggacgtggccgtgtttgatcccgacgtgccgttcctgagttcgtcggcactggcagtcttccagcctgccgtctccagcctgctgcagctcggggagcagccctccgccggcgcccagcagcggctgctggctctgctgcagcagacgtggacgttgatccagaataccaattcgccctccgtggtgatcaacaccctgatcgacgctgggttcacgccctcgcactgcacgcactacctttcggccctggaggggtttctggcggcgggcgtccccgcgcggacgcccgccggccacggactcggcgaagtccagcagctctttgggtgcattgccctcgcggggtcgaacgtgtttggtctggcgcgggaatacgggtactatgccaactacgtaaaaactttcaggcgggtccagggcgccagcgagcacacgcacgggcggctctgcgaggcggtcggcctgtcggggggcgttctgagccagacgctggcgcgtatcatgggtccggccgtgccgacggaacatctggcgagcctgcggcgggcgctcgtgggggagtttgagacggccgagcgccgctttagttccggtcaacccagccttctccgcgagacggcgctcatctggatcgacgtgtatggtcagacccactgggacatcacccccaccaccccggccacgccgctgtccgcgcttctccccgtcgggcagcccagccacgccccctctgtccacctggccgcggcgacccagatccgcttccccgccctcgagggcattcaccccaacgtcctcgccgacccgggcttcgtcccctacgttctggccctggtggtcggggacgcgttgagggccacgtgtagcgcggcctaccttccacgcccggtcgagttcgccctgcgtgtgttggcctgggcccgggactttgggctgggctatctccccacggtcgagggccatcgcaccaaactgggcgcgctgattaccctcctcgaaccggccgcccggggcggcctcggccccactatgcagatggccgacaacatagagcagctgctccgggagctgtacgtgatctccaggggtgccgtcgagcagctgcggcccctggtccagctgcagccccccccgccccccgaggtgggcaccagcctcctgttgattagcatgtacgccctggccgcccggggggtgctgcaggacctcgccgagcgcgcagaccccctgattcgccaactggaggacgccatcgtgctgctgcggctgcacatgcgcacgctctccgcctttttcgagtgtcggttcgagagcgacgggcgccgcctgtatgcggtggtcggggacacgcccgaccgcctggggccctggccccccgaggccatgggggacgcggtgagtcagtactgcagcatgtatcacgacgccaagcgcgcgctggtcgcgtccctcgcgagcctgcgttccgtcatcaccgaaaccacggcgcacctgggggtgtgcgacgagctggcggcccaggtgtcgcacgaggacaacgtgctggccgtggtccggcgcgaaattcacgggtttctgtccgtcgtgtccggcattcacgcccgggcgtcgaagctgctgtcgggagaccaggtccccgggttttgcttcatgggtcagtttctagcgcgctggcggcgtctctcggcctgctatcaagccgcgcgcgcggccgcgggacccgagcccgtggccgagtttgtccaggaactccacgacacgtggaagggcctgcagacggagcgcgccgtggtcgtggcgcccttggtcagctcggccgaccagcgcgccgcggccatccgagaggtaatggcgcatgcgcccgaggacgcccccccgcaaagccccgcggccgaccgcgtcgtgcttacgagccgtcgcgacctagaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2026/0097088 A1
(43) Pub. Date: Apr. 9, 2026

(54) MACROPHAGE POLARIZING ONCOLYTIC HERPES SIMPLEX VIRUS FOR CANCER THERAPY

(71) Applicant: Research Institute at Nationwide Children's Hospital, Columbus, OH (US)

(72) Inventors: Chun-Yu Chen, Columbus, OH (US); Pin-Yi Wang, Columbus, OH (US); Timothy P. Cripe, Columbus, OH (US)

(21) Appl. No.: 19/113,359

(22) PCT Filed: Aug. 17, 2023
(86) PCT No.: PCT/US2023/072415

§ 371 Date: Mar. 19, 2025

Related U.S. Application Data
(60) Provisional application No. 63/416,783, filed on Oct. 17, 2022.

(51) Int. Cl. A61K 35/763; A61K 38/17; A61P 35/00; C12N 7/00

(52) U.S. Cl. CPC A61K 35/763; A61K 38/1741; A61P 35/00; C12N 7/00; C12N 2710/16622; C12N 2710/16634; C12N 2710/16671

(57) ABSTRACT

This disclosure relates to a modified oncolytic herpes simplex virus (oHSV) comprising an expression cassette encoding a histidine-rich glycoprotein (HRG), and uses thereof. One promising avenue for the treatment of cancer is oncolytic virotherapy, e.g., oncolytic Herpes Simplex Virus (oHSV) which utilizes genetically modified viruses to selectively target and lyse cancer cells while sparing the normal cells. Oncolytic virotherapy is a safe and effective immunotherapeutic platform for different types of cancers.

### CROSS-REFERENCE TO RELATED PATENT APPLICATION

This application claims the benefit under 35 U.S.C. § 119(e) of U.S. Provisional Application No. 63/416,783, filed Oct. 17, 2022, the entire contents of which are incorporated herein by reference.

### STATEMENT OF GOVERNMENT SUPPORT

This invention was made with government support under W81XWH-18-1-0324 awarded by the Department of Defense. The government has certain rights in the invention.

### SEQUENCE LISTING

The instant application contains a Sequence Listing which has been submitted electronically in XML format and is hereby incorporated by reference in its entirety. Said XML copy, created on Jul. 25, 2023, is named 106887-9160.xml and is 41,583 bytes in size.

### BACKGROUND

The present invention relates generally to the field of cancer therapy.

Despite the tremendous advances in the treatment of localized cancers, overall outcomes for patients with relapse or disseminated diseases remain poor. In addition, pediatric cancer patients often suffer side effects from chemotherapy and radiation therapy, necessitating the development of more effective but safe therapies. Thus, a need exists in the art for safe and effective therapies for these difficult to treat cancers. This disclosure satisfies this need and provides related advantages as well.

### SUMMARY OF THE DISCLOSURE

One promising avenue for the treatment of cancer is oncolytic virotherapy, e.g., oncolytic Herpes Simplex Virus (oHSV) which utilizes genetically modified viruses to selectively target and lyse cancer cells while sparing the normal cells. Oncolytic virotherapy is a safe and effective immunotherapeutic platform for different types of cancers. Oncolytic viruses selectively destroy cancer cells via direct tumor cell lysis, but spare the normal surrounding cells. In addition, oncolytic virotherapy induces cytotoxic cytokines and T-cell chemokines to promote antitumor T cell responses, which are crucial for systemic and long-term antitumor protection.

Although oHSV therapy showed promising results in preclinical and clinical studies, not all the tumor models respond well to oHSV treatment; and often times oHSV treatment only delayed tumor growth with few long-term survivors.

Few human histidine-rich glycoprotein (hHRG)-expressing vectors including Adenovirus or Adeno-associated viruses (AAV) are commercially available but none of them are ideal for clinical use. These viruses don't replicate well in tumor and may cause systemic side effects when infusing adeno-associated virus comprising hHRG (AAV-hHRG) or adenovirus comprising hHRG (Ad-hHRG) systemically. Using oHSV to express hHRG is novel and safe. oHSV only replicates well in tumor cells but not normal cells which allows us to express abundant therapeutic protein in tumor without causing unwanted systemic side effects.

This disclosure exploits macrophage polarization (e.g., M1-like vs. M2-like) to enhance oncolytic virotherapy and ultimately achieve better patient outcomes. The instant disclosure provides an improved virotherapy by promoting the polarization of antitumorigenic M1 macrophages from protumorigenic M2-like macrophages in tumor using a novel oncolytic herpes variant (oHSV-hHRG) expressing human HRG (hHRG).

In one aspect, this disclosure provides a modified oncolytic herpes simplex virus (oHSV) comprising an expression cassette encoding a histidine-rich glycoprotein (HRG), optionally wherein the oHSV comprises an attenuated oHSV.

Another aspect of the disclosure is directed to a population of modified oncolytic herpes simplex viruses, comprising a plurality of modified oHSVs, wherein the oHSVs are selected from oHSVs of the instant disclosure, wherein the modified oHSVs are the same or different from each other.

Another aspect of the disclosure is directed to an isolated host cell comprising the modified oHSV of the instant disclosure and/or a polynucleotide encoding the oHSV of this disclosure.

Another aspect of the disclosure is directed to an oncolytic herpes simplex virus (oHSV) vector comprising, or consisting essentially of, or consisting of the modified oHSV as disclosed herein.

Another aspect of the disclosure is directed to a method for expressing histidine-rich glycoprotein (HRG) comprising growing a cell comprising, or consisting essentially of, or consisting of one or more of the modified oHSV of the instant disclosure, the population of oHSVs of the instant disclosure, or the oHSV vector of the instant disclosure under conditions that favor expression of HRG.

Another aspect of the disclosure is directed to a method for inhibiting the growth of a cancer cell or treating cancer comprising, or consisting essentially of, or consisting of administering to a subject suffering from the cancer an effective amount of the modified oHSV of the instant disclosure, the population of oHSVs of the instant disclosure, or the oHSV vector of the instant disclosure.

Another aspect of the disclosure is directed to a method for changing the polarization of macrophages from M2-like to M1-like in a subject comprising, or consisting essentially of, or yet further consisting of administering to the subject one or more of the modified oHSV of the instant disclosure, the population of oHSVs of the instant disclosure, or the oHSV vector of the instant disclosure.

Kits to perform the methods containing the oHSV vectors and compositions as described herein are further provided.

### BRIEF DESCRIPTION OF THE DRAWINGS

FIGS. 1A-1F show schematic diagrams of generating oHSV-hHRG and corresponding control viruses. (A) Wild type HSV. (B) 17Δ34.5-hHRG. (C) 17Δ34.5-SecAhHRG. (D) 17Δ34.5-GFP, a control virus for 17Δ34.5-hHRG and 17Δ34.5-hHRG. (E) Mut3ΔICP6-SecAhHRG. (F) Mut3ΔICP6-GFP, a control virus for Mut3ΔICP6-SecAhHRG.

FIG. 1G shows that SecA peptide enhances HRG secretion in a human tumor cell line. A673 tumor cells were infected with 17Δ34.5-hHRG or 17Δ34.5-SecAhHRG viruses at MOI. 0.1, 1 or 10. Supernatants were collected 24 hours after infection for hHRG ELISA analysis.

FIGS. 2A-2B show expression of human histidine-rich glycoprotein (HRG) in herpes simplex virus (HSV)-HRG infected cells. (A) Western blot analysis of BHK-21 cells only (Mock), infected with parental virus (17Δ34.5-GFP) or infected with HRG-expressing HSV (17Δ34.5-SecAhHRG). Human HRG can only be detected in HSV-HRG infected lysate while GAPDH housekeeping control expression is relative abundant across the samples. (B) ELISA analysis of hHRG secretion. BHK cells or A673 tumor cells were infected with 17Δ34.5-GFP or 17Δ34.5-SecAhHRG viruses at MOI=1. Supernatants were collected 24 hours after infection for hHRG ELISA analysis. MOI, multiplicity of infection.

FIGS. 3A-3B show 17Δ34.5-SecAhHRG (HSV-HRG) and 17Δ34.5-GFP (HSV) display similar virus activities in vitro. (A) In vitro virus replication assays. M3-9-M or 76-9 cells were infected with HSV (gray column) or HSV-HRG (black column) virus at MOI=0.01. At the indicated time points, infected cells were collected and viral titers were determined by plaque assays. (B) Cytotoxicity assays. M3-9-M or 76-9 cells were infected with HSV (◯) or HSV-HRG (●) at the indicated MOIs. Cell viabilities were evaluated by MTS-assay three days post virus infection. Pfu: plaque forming unit.

FIG. 4 shows that A673, human Ewing's sarcoma cell line, exhibits similar sensitivity to 17Δ34.5-SecAhHRG (HSV-HRG) and 17Δ34.5-GFP (HSV). A673 cells were infected with HSV (◯) or HSV-HRG (●) at the different MOIs. Cell viabilities were evaluated by MTS-assay three days post virus infection.

FIGS. 5A-5K show 17Δ34.5-SecAhHRG (HSV-HRG) and 17Δ34.5-GFP (HSV) induce similar level of chemokine and cytokine expressions in murine RMS cell lines. M3-9-M or 76-9 cells were infected with HSV-HRG (●) or HSV (▴) at the MOI=1 overnight. Mock-infected (▪) cells were harvested as a control. RNA samples were prepared for quantitative RT-PCR. Quantitative RT-PCT results are as shown for (A) Hrg, (B) Gc, (C) Ccl-2, (D) Tnf-a, (E) Cxcl-10, (F) Ccl-5, (G) Ccl-17, (H) Tgf-b, (I) IL-10, (J) Cxcl-1, and (K) Cxcl-2. Data are presented as relative RNA expression to gapdh.

FIGS. 6A-6I show 17Δ34.5-SecAhHRG (HSV-HRG) and 17Δ34.5-GFP (HSV) induce similar chemokine and cytokine expressions in A673 cell line. A673 cells were infected with HSV-HRG (●) or HSV (▴) at the MOI=0.1 overnight. Mock-infected (▪) cells were harvested as a control. RNA samples were prepared for quantitative RT-PCR. (A) Gc, (B) hHrg, (C) hCcl-2, (D) hCcl5, (E) hTgfM1, (F) hIL-10, (G) hTNFa, (H) h118, and (I) hCxc110. Data are presented as relative RNA expression to gapdh.

FIG. 7 shows that 17Δ34.5-SecAhHRG (HSV-HRG) induces more M1 macrophage polarization in vitro. M0, M1 or M2 macrophages derived from murine bone marrow cells were infected with 17Δ34.5-SecAhHRG (HSV-HRG) or 17Δ34.5-GFP (HSV) at the MOI=1 overnight. Cells were harvested and stained with M1 marker MHC II.

FIGS. 8A-8G show changes of immune cell infiltrating profile in M3-9-M tumors upon HSV-HRG treatment. Tumor-bearing mice were intratumorally treated with PBS (▪), 10⁸ pfu of 17Δ34.5-GFP (HSV) (▴) or 17Δ34.5-SecAhHRG (HSV-HRG) (●) every other day for three doses. Immune cell infiltrates in tumors were evaluated by flow cytometry analyses 72 hours after the last dose of the virus injection. (A) MBC II^{hi} M1, (B) CD206^{hi} M2, (C) CD8+ T cells, (D) CD4+ T cells, (E) Treg, and (F) CD8+/Treg ratio.

FIGS. 9A-9E show changes of macrophage polarization in M3-9-M tumors upon HSV-HRG treatment. Tumor-bearing mice were intratumorally treated with PBS (▪), 10⁸ pfu 17Δ34.5-GFP (HSV) (▴) or 17Δ34.5-SecAhHRG (HSV-HRG) (●) every other day for three doses. F4/80+ macrophages were enriched from tumors using Miltenyi™ antoMACS™ cell separator three days after the last dose of the virus treatment. M1- and M2-related genes were analyzed by quantitative RT-PCR. Results for (A) Cxcl10, (B) Tnfa, (C) Cd206, (D) Ccl22, and (E) Arg1.

FIGS. 10A-10C show HRG enhances oHSV virotherapy in A673 tumor model. (A) Schematic illustrates the dosing regimens for mice bearing subcutaneous A673 tumors. Female nude mice were implanted with 5×10^{6 Δ673} cells and treated with two intratumoral (i.tu.) injections of 10⁷ pfu of 17Δ34.5-GFP (HSV) (◯), 17Δ34.5-SecAhHRG (HSV-HRG) (●) or PBS (♦). (B) Tumor volumes of mice treated with PBS (solid line), HSV (dot line) and HSV-HRG (dashed line) were measured twice a week. (C) Kaplan-Meier survival curves for each treatment group.

FIGS. 11A-11C show Mut3ΔICP6-SecAhHRG further enhances hHRG secretion compared to 17Δ34.5-SecAhHRG. (A) Western blot analysis of hHRG protein from BHK cells infected with 17Δ34.5-SecAhHRG or Mut3ΔICP6-SecAhHRG virus at the MOl=1 for 24 hours. (B) ELISA analysis of hHRG secretion in vitro. BHK cells were infected with 17Δ34.5-SecAhHRG or Mut3ΔICP6-SecAhHRG virus at the MOI of 0.1, 1 or 1 for one, two or three days. Supernatants were collected for hHRG ELISA analysis. (C) ELISA analysis of hHRG secretion in vivo. A673 tumor-bearing mice were treated with two intratumoral (i.tu.) injections of 10⁷ pfu of Mut3ΔICP6-SecAhHRG or 17Δ34.5-SecAhHRG virus. Treated tumors were harvested two, five or eight days after the last dose of the virus treatment for hHRG ELISA analysis.

FIG. 12 shoes Mut3ΔICP6-SecAhHRG maintains in vitro tumor cell killing activity compared to its control virus, Mut3ΔICP6-GFP. Murine M3-9-M tumor cells were infected with Mut3ΔICP6-GFP (o) or Mut3ΔICP6-SecAhHRG (●) at the indicated MOIs. Cell viabilities were evaluated by MTS-assay three days post virus infection.

FIG. 13 shows the map of 032609delGFPhHRG vector for use to make oHSV clone 17Δ34.5hHRG with native human HRG putative signal peptide. SEQ ID NO: 4 shows the whole vector sequence for this clone and the map shows the annotation of the components of the sequence. CMV promoter sequence: SEQ ID NO: 5. The putative signal sequence (shown as “signal peptide” in the vector map): SEQ ID NO: 6. hHRG encoding sequence (without signal sequence): SEQ ID NO: 7. Polyadenylation signal: SEQ ID NO: 8.

FIG. 14 shows the map of 032608delGFPSecAhHRG vector for use to make oHSV clone 17Δ34.5SecAhHRG with human HRG secrecon signal peptide (SecA). SEQ ID NO: 9 shows the whole vector sequence for this clone and the map shows the annotation of the components of the sequence. CMV promoter sequence: SEQ ID NO: 5. The Kozak sequence: SEQ ID NO: 10. The SecA signal sequence: SEQ ID NO: 11. hHRG encoding sequence (without signal sequence): SEQ ID NO: 7. Polyadenylation signal: SEQ ID NO: 8.

FIG. 15 shows the map of pCK1061DICP6SecAhHRG vector for use to make oHSV clone Mut3ΔICP6SecAhHRG with human HRG secrecon signal peptide (SecA). SEQ ID NO: 12 shows the whole vector sequence for this clone and the map shows the annotation of the components of the sequence. CMV promoter sequence: SEQ ID NO: 5. The Kozak sequence: SEQ ID NO: 10. The SecA signal sequence: SEQ ID NO: 11. hHRG encoding sequence (without signal sequence): SEQ ID NO: 7. Polyadenylation signal: SEQ ID NO: 8.

### DETAILED DESCRIPTION

### Definitions

As it would be understood, the section or subsection headings as used herein is for organizational purposes only and are not to be construed as limiting and/or separating the subject matter described.

The following definitions assist in defining the meets and bounds of the inventions as described herein.

The term “about” when used before a numerical designation, e.g., temperature, time, amount, concentration, and such other, including a range, indicates approximations which may vary by (+) or (−) 10%, 5% or 1%.

The terms “administering” or “administration” in reference to delivering modified oncolytic herpes simplex virus (oHSV) to a subject include any route of introducing or delivering to a subject the oHSV to perform the intended function. Administration can be carried out by any suitable route, including orally, intranasally, parenterally (intravenously, intramuscularly, intraperitoneally, or subcutaneously), intracranially, intratumorally (itu), or topically. Additional routes of administration include intraorbital, infusion, intraarterial, intracapsular, intracardiac, intradermal, intrapulmonary, intraspinal, intrasternal, intrathecal, intrauterine, intravenous, subarachnoid, subcapsular, subcutaneous, transmucosal, or transtracheal. Administration includes self-administration and the administration by another.

“Comprising” or “comprises” is intended to mean that the compositions, for example media, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

As used herein, the term “modified,” relative to oHSVs, refers to oHSVs that have been altered such that they differ from a naturally occurring oHSV. Non-limiting examples of a modified oHSV include an oHSV that contains a nucleic acid or protein of a type or in an amount different than that found in a naturally occurring oHSV. In some embodiments, the modified oHSV is an attenuated oHSV. In some embodiments, the attenuated oHSV comprises a virus whose neurovirulence gene RL1/γ₁34.5 (which encodes ICP34.5 protein) and/or large subunit of ribonucleotide reductase gene UL39 (which encodes ICP6 protein), were replaced by hHRG gene (in attenuated experimental oHSV) or GFP (attenuated control oHSV).

As used herein, the term “detectable label” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, (3-galactosidase, glucose6 phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S, ⁸⁹Zr or ¹²⁵I.

The term “detectable label” also includes sequences conjugated to the polynucleotide that will provide a signal upon expression of the inserted sequences, such as green fluorescent protein (GFP) and the like. The detectable label may be detectable by itself (e.g., radioisotope labels or fluorescent labels) or, in the case of an enzymatic label, may catalyze chemical alteration of a substrate compound or composition which is detectable. The detectable labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected, or it may be quantified. A response that is simply detected generally comprises, alternatively consists essentially of, or yet further consists of a response whose existence merely is confirmed, whereas a response that is quantified generally comprises, alternatively consists essentially of, or yet further consists of a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include but are not limited to bioluminescence and chemiluminescence. Detectable luminescence response generally comprises, alternatively consists essentially of, or yet further consists of a change in, or an occurrence of, a luminescence signal. Suitable methods and luminophores for luminescent labeling assay components are known in the art and described for example in Haugland, Richard P. (1996) Handbook of Fluorescent Probes and Research Chemicals (6th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

As used herein, the term “purification label” refers to at least one marker that can be used for purification or identification. A non-exhaustive list of such markers includes His, lacZ, GST, maltose binding protein, NusA, BCCP, c-myc, CaM, FLAG, GFP, YFP, cherry, thioredoxin, poly (NANP), V5, Snap, HA, chitin binding protein, Softag 1, Softag 3, Strep or S protein. Suitable direct or indirect fluorescent labels include FLAG, GFP, YFP, RFP, dTomato, cherry, Cy3, Cy 5, Cy 5.5, Cy 7, DNP, AMCA, biotin, digoxin, Tamra, texas red, rhodamine, Alexa fluorescence, FITC, TRITC, or any other fluorescent dye or hapten.

As used herein, an “expression cassette” refers to a gene expression system containing all the necessary elements required for expression of the target polypeptide (e.g., HRG). The gene expression system normally includes the following elements: promoter, gene sequences encoding polypeptide and terminator, which are operably linked. Moreover, coding sequences of signal peptide may be optionally included.

As used herein, “operably linked” refers to a functional arrangement of two or more nucleic acid region or nucleic acid sequences. For example: a promoter region is positioned in certain specific positions with respect to the target nucleic acid sequences, such that the promoter region directs the transcription of the nucleic acid sequences, and the promoter region is “operably linked” to the nucleic acid sequences.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has, or is diagnosed of having, or is suspected of having, or is at risk of having a disease, such as a cancer.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

When the disease is cancer, the following clinical endpoints are non-limiting examples of treatment: (1) elimination of a cancer in a subject or in a tissue/organ of the subject or in a cancer loci; (2) reduction in tumor burden (such as number of cancer cells, number of cancer foci, number of cancer cells in a foci, size of a solid cancer, concentrate of a liquid cancer in the body fluid, and/or amount of cancer in the body); (3) stabilizing or delay or slowing or inhibition of cancer growth and/or development, including but not limited to, cancer cell growth and/or division, size growth of a solid tumor or a cancer loci, cancer progression, and/or metastasis (such as time to form a new metastasis, number of total metastases, size of a metastasis, as well as variety of the tissues/organs to house metastatic cells); (4) less risk of having a cancer growth and/or development; (5) inducing an immune response of the patient to the cancer, such as higher number of tumor-infiltrating immune cell, higher number of activated immune cells, or higher number cancer cell expressing an immunotherapy target, or higher level of expression of an immunotherapy target in a cancer cell; (6) higher probability of survival and/or increased duration of survival, such as increased overall survival (OS, which may be shown as 1-year, 2-year, 5-year, 10-year, or 20-year survival rate), increased progression free survival (PFS), increased disease free survival (DFS), increased time to tumor recurrence (TTR) and increased time to tumor progression (TTP). In some embodiments, the subject after treatment experiences one or more endpoints selected from tumor response, reduction in tumor size, reduction in tumor burden, increase in overall survival, increase in progression free survival, inhibiting metastasis, improvement of quality of life, minimization of drug-related toxicity, and avoidance of side-effects (e.g., decreased treatment emergent adverse events). In some embodiments, improvement of quality of life includes resolution or improvement of cancer-specific symptoms, such as but not limited to fatigue, pain, nausea/vomiting, lack of appetite, and constipation; improvement or maintenance of psychological well-being (e.g., degree of irritability, depression, memory loss, tension, and anxiety); improvement or maintenance of social well-being (e.g., decreased requirement for assistance with eating, dressing, or using the restroom; improvement or maintenance of ability to perform normal leisure activities, hobbies, or social activities; improvement or maintenance of relationships with family). In some embodiments, improved patient quality of life that is measured qualitatively through patient narratives or quantitatively using validated quality of life tools known to those skilled in the art, or a combination thereof. Additional non-limiting examples of endpoints include reduced hospital admissions, reduced drug use to treat side effects, longer periods off-treatment, and earlier return to work or caring responsibilities. In one aspect, prevention or prophylaxis is excluded from treatment.

In certain embodiments, the terms “disease” “disorder” and “condition” are used interchangeably herein, referring to a cancer, a status of being diagnosed with a cancer, or a status of being suspect of having a cancer. “Cancer,” which is also referred to herein as “tumor”, is a known medically as an uncontrolled division of abnormal cells in a part of the body, benign or malignant. In one embodiment, cancer refers to a malignant neoplasm, a broad group of diseases involving unregulated cell division and growth, and invasion to nearby parts of the body. Non-limiting examples of cancers include carcinomas, sarcomas, leukemia and lymphoma, e.g., colon cancer, colorectal cancer, rectal cancer, gastric cancer, esophageal cancer, head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas (such as rhabdomyosarcoma (RMS), Ewing's sarcoma), carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. As used herein, an advanced cancer refers to a cancer that had progressed after receiving one or more of: the first line therapy, the second line therapy, or the third line therapy.

The term “contacting” means direct or indirect binding or interaction between two or more. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity can determined by incorporating them into clustalW (available at the web address:genome.jp/tools/clustalw/, last accessed on Jan. 13, 2017).

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure. In some embodiments, BLAST (accessible at blast.ncbi.nlm.nih.gov/Blast.cgi) or Clustal Omega (accessible at www.ebi.ac.uk/Tools/msa/clustalo/) are used in determining the identity. In further embodiments, default setting is applied.

As used herein, the term “pharmaceutically acceptable carrier” refers to any of the standard pharmaceutical carriers such as sterile solutions, tablets, coated tablets, and capsules. Typically such carriers contain excipients such as starch, milk, sugar, certain types of clay, gelatin, stearic acids or salts thereof, magnesium or calcium stearate, talc, vegetable fats or oils, gums, glycols, or other known excipients. Such carriers may also include flavor and color additives or other ingredients. Examples of pharmaceutically acceptable carriers include, but are not limited to, the following: water, saline, buffers, inert, nontoxic solids (e.g., mannitol, talc). Compositions comprising such carriers are formulated by well-known conventional methods. Depending on the intended mode of administration and the intended use, the compositions may be in the form of solid, semi-solid, or liquid dosage forms, such, for example, as powders, granules, crystals, liquids, suspensions, liposomes, pastes, creams, salves, etc., and may be in unit-dosage forms suitable for administration of relatively precise dosages.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

A combination as used herein intends that the individual active ingredients of the compositions are separately formulated for use in combination and can be separately packaged with or without specific dosages. The active ingredients of the combination can be administered concurrently or sequentially.

An “effective amount” intends an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks.

“Therapeutically effective amount” of an agent refers to an amount of the agent that is an amount sufficient to obtain a pharmacological response; or alternatively, is an amount of the agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose and may occur only after administration of a series of doses. Thus, a therapeutically effective amount may be administered in one or more administrations.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, lentiviruses, adenoviruses, herpesvirus, bacculoviruses, modified bacculoviruses, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

As used herein, the phrase “derived from” means isolated from, purified from, or engineered from, or any combination thereof.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a β-actin promoter, a phosphoglycerol kinase (PGK) promoter, a U6 promoter, or an EF1 promoter. In some embodiments, the promoter is a chicken β-actin (“CBA”) promoter.

In some embodiments, the promoter that an inducible promoter. In a specific embodiment, the promoter is an inducible tetracycline promoter. The Tet-Off and Tet-On Gene Expression Systems give researchers ready access to the regulated, high-level gene expression systems described as Tet-Off and Tet-On. In the Tet-Off system, gene expression is turned on when tetracycline (Tc) or doxycycline (Dox; a Tc derivative) is removed from the culture medium. In contrast, expression is turned on in the Tet-On system by the addition of Dox. Both systems permit gene expression to be tightly regulated in response to varying concentrations of Tc or Dox. Maximal expression levels in Tet systems are very high and compare favorably with the maximal levels obtainable from strong, constitutive mammalian promoters such as CMV. Unlike other inducible mammalian expression systems, gene regulation in the Tet Systems is highly specific, so interpretation of results is not complicated by pleiotropic effects or nonspecific induction. In E. co/i, the Tet repressor protein (TetR) negatively regulates the genes of the tetracycline-resistance operon on the Tn10 transposon. TetR blocks transcription of these genes by binding to the tet operator sequences (tetO) in the absence of Tc. TetR and tetO provide the basis of regulation and induction for use in mammalian experimental systems. In the Tet-On system, the regulatory protein is based on a “reverse” Tet repressor (rTetR) which was created by four amino acid changes in TetR (Hillen & Berens, Mechanisms underlying expression of Tn10 encoded tetracycline resistance. Annu Rev Microbiol. 1994; 48: 345-69; Gossen et al., Transcriptional activation by tetracyclines in mammalian cells. Science. 1995 Jun. 23; 268(5218):1766-9). The resulting protein, rtTA (reverse tTA also referred to tetracycline activator protein), is encoded by the pTet-On regulator plasmid.

Additional non-limiting exemplary promoters with certain target specificity are provided herein below including but not limited to CMV, EF1a, SV40, PGK1 (human or mouse), P5, Ubc, human beta actin, CAG, TRE, UAS, Ac5, Polyhedrin, CaMKIIa, Gal1, TEF1, GDS, ADH1, CaMV35S, Ubi, H1, U6, and Alpha-1-antitrypsin. Synthetically-derived promoters may be used for ubiquitous or tissue specific expression. Further, virus-derived promoters, some of which are noted above, may be useful in the methods disclosed herein, e.g., CMV, HIV, adenovirus, and AAV promoters. In some embodiments, the promoter is coupled to an enhancer to increase the transcription efficiency.

An enhancer is a regulatory element that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

The term “tumor-specific promoter or tissue-specific promoter” as used herein means a promoter permitting expression of a gene, which is under control of the promoter, specifically in a desired tumor cell or tissue. Non-limiting examples of tissue-specific promoters that can be used in the invention include a prostate-specific antigen (PSA) promoter, a prostate-specific membrane antigen (PSMA) promoter, a casein promoter, an IgG promoter, a chorionic embryonic antigen promoter, an elastase promoter, a porphobilinogen deaminase promoter, an insulin promoter, a growth hormone factor promoter, an acetylcholine receptor promoter, an alcohol dehydrogenase promoter, and an α or β globin promoter.

Non-limiting examples of tumor-specific promoters to be used in the present invention include the telomerase reverse transcriptase promoter, the glial fibrillary acidic protein promoter, an E2F promoter; a survivin promoter, a COX-2 promoter, an EGD-2 promoter; an ELF-1 promoter; a hypoxia-specific promoter; a carcinoembryonic antigen promoter, and the stromelysin 3 promoter.

The term “herpes simplex virus” or “HSV” as used herein means a herpes simplex virus that produces the effect of the present invention, which includes a wild type or mutant herpes simplex virus. In one embodiment, the mutant non-natural HSV is obtained by mutating or modifying any of the genes of wild-type HSV or by inserting any of exogenous genes. The serum type of HSV comprises, alternatively consists essentially of, or yet further consists of a type 1 HSV (or HSV-1) or a type 2 HSV (or HSV-2). The HSV-1 is an enveloped, double-stranded DNA virus. In one embodiment, the HSV-1 can infect a human cell. In another embodiment, a sequence, a gene or multiple genes can be incorporated to the HSV-1. The size of incorporated sequence can be approximate 1 base, 5 bases, 10 bases, 100 bases, 1 kb, 10 kb, 100 kb, or 150 kb. HSV-1 can induce cell lysis at a relatively low multiplicity of infection (MOI), and its proliferation can be inhibited by anti-viral drugs. In one embodiment, the HSV viral DNA stays outside the chromosomes without being incorporated into the genome of host cells. The HSV-1 can encompass a variety of strains (e.g., KOS and McKrae). See Wang et al., (2013) Virus Res. 173(2):436-440. In one embodiment, the HSV-1 is an HSV-1 KOS strain. In another embodiment, the HSV-1 is an HSV-1 McKrae strain.

In some embodiments, the HSV (or oHSV) is attenuated. As used herein, “attenuated HSV (or oHSV)” refers to HSV (or oHSV) whose toxicity has been reduced by modification or mutation.

There are several attenuated HSV mutants, for example, 17TermA HSV and rRp450 HSV. The term “17TermA HSV” refers to mutant HSV-1 virus that comprises the entire ICP34.5 gene, but with a termination codon inserted before 100 bp of coding region, resulting in early termination of protein expression and expression of a 30 amino acid truncated protein. The 17TermA HSV mutant displays a growth defect because of the truncated ICP34.5 protein. See Orvedahl et al., (2007) Cell Host & Microbe, 1:1, 23-25. The term “rRp450” refers to an attenuated herpes simplex 1 vector deficient in the viral-encoded ribonucleotide reductase or ICP6. See Aghi M et al., (1999) Cancer Res., 59(16):3861-5. In some embodiments, hHRG expressing attenuated oHSVs do not have ICP34.5 (17Δ34.5-SecAhHRG) and/or ICP6 (Mut3ΔICP6SecAhHRG).

The HSV genome encodes multiple virulence proteins, which include but are not limited to glycoprotein E (“gE”), Infected Cell Protein 0 (“ICP0”), Infected Cell Protein 6 (“ICP6”), DNA packaging terminase subunit 1, Infected Cell Protein 8 (“ICP8”), and Infected Cell Protein 34.5 (“ICP34.5”). An exemplary HSV1 genome can be found at NCBI Reference Sequence: NC_001806.2.

The term “gE-encoding gene” refers to a gene or its DNA fragment encoding a gE protein. An exemplary gE-encoding gene can be identified at positions 33-2555 of the HSV-1 genome sequence at NCBI Reference Sequence: NC_001806.2. The term “ICP6 protein” refers to an infected cell protein 6 encoded by the HSV genome. ICP6 is a subunit of ribonucleotide reductase (“RR”) and a key enzyme for nucleotide metabolism and viral DNA synthesis in non-dividing cells.

A “dysfunctional” protein refers to a protein that has an impaired or no function of the original protein. In one embodiment, a dysfunctional protein is caused by deletion or substitution in the coding sequences. For example, with a dysfunctional ICP6 gene, be deletion or inactivation, HSV cannot replicate in normal non-dividing cells. In actively dividing cells with increased RR activity, however, the deficient enzyme activity of the virus is compensated, enabling the virus to replicate.

The term “deletion or inactivation of a gene” means deletion of the whole or portion of the gene or suppression of expression of the gene through substitution of some bases, modification, insertion of an unnecessary sequence or the like. The deletion or inactivation of the HSV gene (e.g., gE, ICP0, and ICP8) can be conducted by those skilled in the art in a known method or a method based thereon. For example, a method using homologous recombination can be employed. For example, it is possible to divide and inactivate the HSV gene by cloning a DNA fragment containing a portion of the HSV gene and a sequence unrelated to the HSV gene in a suitable plasmid vector and then introducing it into HSV to cause homologous recombination in some region of the HSV gene. Alternatively, the mutation or deletion of an HSV gene can be caused by spontaneous mutation in the viral passage.

As used herein, the term “eukaryotic cell” refers to any animal or plant cell having a definitive nucleus. Eukaryotic cells of animals include cells of vertebrates such as mammals, and cells of invertebrates such as insects. Examples of eukaryotic cells of plants include yeast cells, and algae cells. Eukaryotic cells can also comprise antibody producing cells, such as hybridoma. The term “prokaryotic cell” refers to a cell of a prokaryotic organism that lacks a definitive nucleus. Examples of prokaryotic cells can include, but are not limited to, the genus Escherichia, Bacillus or Lactococcus. Some examples of prokaryotic cell species from these genera are Escherichia coli, Bacillus subtilis or Lactococcus lactis.

As used herein, the term “M0 macrophages” refers to uncommitted macrophages, the term “M1 macrophages” refers to activated pro-inflammatory macrophages and the term “M2 macrophages” refers to activated anti-inflammatory macrophages. See, e.g., Kishore, Amit, and Martin Petrek. Frontiers in immunology 12 (2021): 678457, which is incorporated herein in its entirety. Macrophage polarization from M0 to M1 or M2 can be measured by microscopy (based on morphological changes), RT-qPCR or flow cytometry, as described in the art. See, e.g., Bertani, Francesca R., et al., Scientific reports 7.1 (2017): 1-9, which is incorporated herein in its entirety.

Modified Oncolytic Herpes Simplex Virus (oHSV)

The efficacy of oHSVs against tumors is derived from direct cell killing (lytic phase) and enhancing anticancer immunity (immune phase). These viruses have been constructed in various ways to selectively target cancer cells. To achieve cancer selectivity, the most common mutation is deletion of the neurovirulence gene, γ₁34.5/RL1. Expression of ICP34.5, encoded by γ₁34.5/RL1, is essential for HSV-1 to counteract the host cell anti-viral protein kinase RNA-activated (PKR)-pathway that normally phosphorylates e-IF2α in response to virus infection and stops protein translation. ICP34.5 redirects cellular protein phosphatase-1 (PP1) to dephosphorylate e-IF2a, allowing productive virus replication.

Many cancer cells are defective in the PKR response, and therefore support the replication of HSV vectors including γ₁34.5-null mutants. Some vectors are constructed by mutating critical metabolic viral genes such as ribonucleotide reductase (RR, the large subunit of which is encoded by the ICP6/UL39 gene). Since many malignant cells have increased expression and activity of RR, ICP6-null mutants selectively replicate in highly proliferating cancer cells because of the large ribonucleotide pools present.

An aspect of the disclosure is directed to a modified oncolytic herpes simplex virus (oHSV) comprising, or consisting essentially of, or consisting of an expression cassette encoding a histidine-rich glycoprotein (HRG), optionally wherein the oHSV comprises an attenuated oHSV.

In some embodiments, the HRG comprises an amino acid sequence with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: 1. In a further aspect, the HRG comprises, or consists essentially of, or consists of SEQ ID NO: 1.

In some embodiments, the HRG comprises an amino acid sequence with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: 2. In a further aspect, the HRG comprises, or consists essentially of, or consists of SEQ ID NO: 2.

In some embodiments, the HRG is fused to a SecA signal peptide. In some embodiments, the SecA signal peptide comprises, or consists essentially of, or consists of SEQ ID NO: 3 or an equivalent thereof with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: 3. In some embodiments, the SecA signal peptide is encoded by a nucleic acid that comprises, or consists essentially of, or consists of SEQ ID NO: 11 or with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: 11 that also encodes SEQ ID NO.: 3 or the equivalent thereof

In some embodiments, the modified oHSV further comprises a mutation in a virulence gene that is one or more from the group of: (a) a glycoprotein E (“gE”)-encoding gene, (b) an Infected Cell Protein 0 (“ICP0”)-encoding gene, (c) a DNA packaging terminase subunit 1-encoding gene, (d) an ICP8-encoding gene, or (e) an ICP34.5-encoding gene as described in WO2020/186238, which is incorporated herein in its entirety. In one embodiment, the oHSV further comprises, alternatively consists essentially of, or consists of a gene encoding a dysfunctional ICP34.5 protein and/or a gene encoding a dysfunctional ICP6 protein. In some embodiments, “a gene encoding a dysfunctional ICP34.5 protein” comprises deletion of part or all of the gene encoding ICP34.5. In some embodiments, “a gene encoding a dysfunctional ICP34.5 protein” comprises mutation in the gene encoding ICP34.5 such that no functional ICP34.5 protein is produced (e.g., an early stop codon, or a mutation that causes a frameshift). In some embodiments, “a gene encoding a dysfunctional ICP6 protein” comprises deletion of part or all of the gene encoding ICP6. In some embodiments, “a gene encoding a dysfunctional ICP6 protein” comprises mutation in the gene encoding ICP6 such that no functional ICP6 protein is produced (e.g., an early stop codon, or a mutation that causes a frameshift).

In some embodiments, the oHSV comprises oHSV wherein both γ34.5 genes are deleted and the oHSV comprises an hHRG with a SecA signal sequence (the 17Δ34.5SecAhHRGvariant). In some embodiment, the 17Δ34.5SecAhHRG variant comprises, or consists essentially of, or consists of SEQ ID NO: 9 or an equivalent thereof with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: 9. In some embodiments, the oHSV comprises an oHSV wherein the ICP6 are deleted and the oHSV comprises an hHRG with a SecA signal sequence (the Mut3ΔICP6SecAhHRG variant). In some embodiment, the Mut3ΔICP6SecAhHRG variant comprises, or consists essentially of, or consists of SEQ ID NO: 12 or an equivalent thereof with at least 80% (e.g., 80%, 81%, 82%, 83%, 84%, 85%, 86%, 87%, 88%, 89%0, 90%, 91%, 92%9, 93%, 94%, 95%, 96%, 97%, 98%, 99% or more) sequence identity to SEQ ID NO: 12.

In some embodiments, the oHSV further comprises a Kozak sequence, optionally wherein the Kozak sequence comprises, or consists essentially of, or consists of SEQ ID NO: 10 or an equivalent thereof with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: 10.

some embodiments, the oHSV further comprises a polyadenylation signal, wherein the polyadenylation signal comprises, or consists essentially of, or consists of SEQ ID NO: 8 or an equivalent thereof with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: 8.

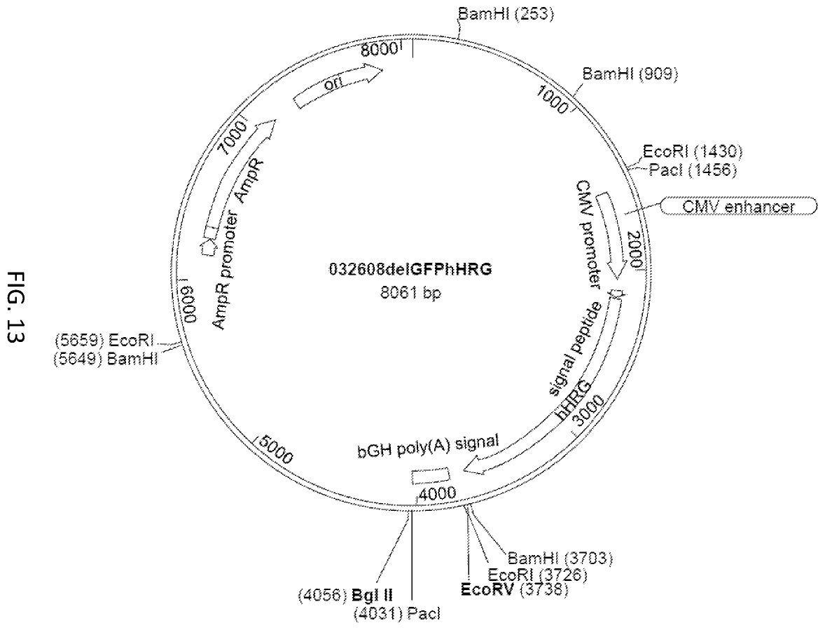

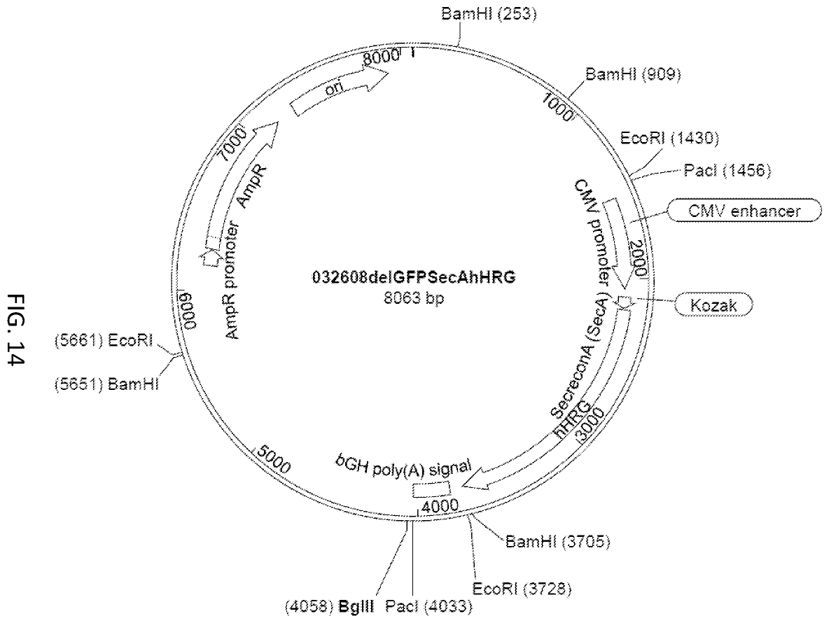

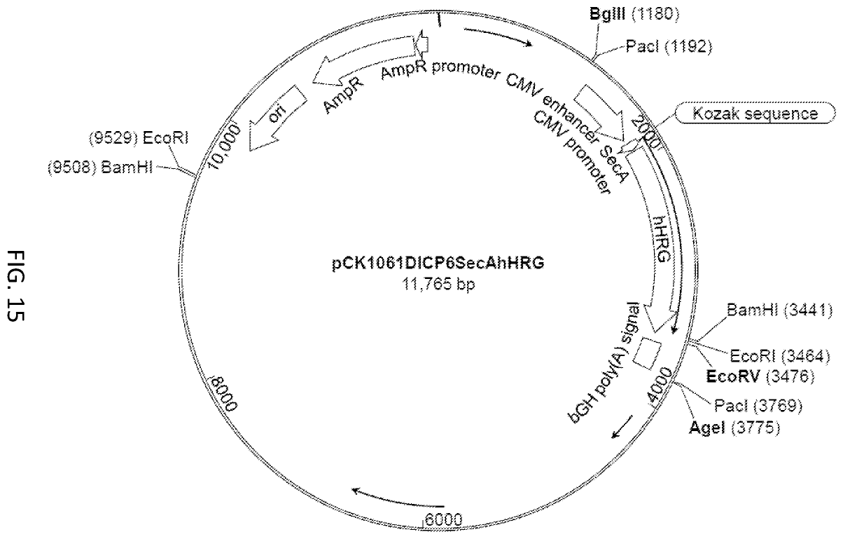

In another aspect, the OHSV comprises, or consists essentially of, or yet further consist of the oHSV shown any one of FIGS. 13-15, or an equivalent thereof with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: to the oHSV shown in FIGS. 13-15, respectively.

In some embodiments, the oHSV comprises HSV-1 strain or an HSV-2 strain. In some embodiments, the HSV-1 strain is selected from HSV-1 strain F (deposited at American Type Culture Collection (ATCC), www.atcc.org/products/vr-733 under Accession No.: VR-733), strain KOS (deposited at American Type Culture Collection (ATCC), www.atcc.org/products/vr-1493 under Accession No.: VR-1493), strain HF (deposited at American Type Culture Collection (ATCC), www.atcc.org/products/vr-260 under Accession No.: VR-260), strain McIntyre (deposited at American Type Culture Collection (ATCC), www.atcc.org/products/vr-539 under Accession No.: VR-539), or any clinical isolate HSV-1. In some embodiments, the HSV-2 strain is selected from HSV-2 strain G (deposited at American Type Culture Collection (ATCC), www.atcc.org/products/vr-734 under Accession No.: VR-734), strain ATCC-2011-2 (deposited at American Type Culture Collection (ATCC), www.atcc.org/products/vr-1779 under Accession No.: VR-1779), strain MS (deposited at American Type Culture Collection (ATCC), www.atcc.org/products/vr-540 under Accession No.: VR-540), ATCC-2011-4 (deposited at American Type Culture Collection (ATCC), www.atcc.org/products/vr-1781 under Accession No.: VR-1781), or any clinical isolate HSV-2.

Herpes Simplex Virus (HSV) 1 and 2 are members of the Herpesviridae family, which infects humans. The HSV genome contains two unique regions, which are designated unique long (UL) and unique short (Us) region. Each of these regions is flanked by a pair of inverted terminal repeat sequences. There are about 75 known open reading frames. The viral genome has been engineered to develop oncolytic viruses (i.e., viruses that preferentially infect and kill cancer cells) for use in, e.g., cancer therapy. Tumor-selective replication of HSV may be conferred by mutation of the HSV ICP34.5 (also called 734.5) gene (Liu, B. L., et al., Gene therapy 10.4 (2003): 292-303). HSV contains two copies of ICP34.5. Mutants inactivating one or both copies of the ICP34.5 gene are known to lack neurovirulence, i.e., be avirulent/non-neurovirulent and be oncolytic. The oHSV can be reproduced using known recombinant methods, e.g., as shown in Nguyen, Hong-My, et al. (“Growth, Purification, and Titration of Oncolytic Herpes Simplex Virus.” JoVE (Journal of Visualized Experiments) 171 (2021): e62677), Froechlich, Guendalina, et al. “Integrity of the antiviral STING-mediated DNA sensing in tumor cells is required to sustain the immunotherapeutic efficacy of herpes simplex oncolytic virus.” Cancers 12.11 (2020): 3407, and Froechlich, Guendalina, et al. “Generation of a novel mesothelin-targeted oncolytic herpes virus and implemented strategies for manufacturing.” International journal of molecular sciences 22.2 (2021): 477, each of which is incorporated herein in its entirety.

Also provided is a polynucleotide encoding an oHSV of this disclosure.

In some embodiments, the modified oHSV and/or polynucleotide encoding the oHSV of this disclosure further comprises a detectable or purification label, examples of such are known in the art and described herein.

Another aspect of the disclosure is directed to a population of modified oncolytic herpes simplex viruses, comprising a plurality of modified oHSVs, wherein the oHSVs are selected from oHSVs of the disclosure, wherein the modified oHSVs are the same or different from each other. One of skill in the art can produce the population by admixing the modified oHSV in the ratios and selection of components as desired.

Another aspect of the disclosure is directed to a composition comprising the modified oHSV or polynucleotide encoding same and/or the population of the modified oHSV of the instant disclosure. In some embodiments, the composition further comprises a carrier, optionally a pharmaceutically acceptable carrier. One of skill in the art can produce the composition by admixing the modified oHSV in the ratios and selection of components as desired in the carrier, such as a pharmaceutically acceptable carrier.

In some embodiments, the composition further comprises an additional therapeutic agent, optionally an anticancer therapeutic agent. One of skill in the art can produce the composition by admixing the modified oHSV in the ratios and selection of components as desired in the carrier, such as a pharmaceutically acceptable carrier. In some embodiments, the additional therapeutic agent is selected from the group of: 5-fluorouracil, pemetrexed, raltitrexed, nolatrexed, plevitrexed, GS7904L, capecitabine, methotrexate, pralatrexate, CT-900, NUC-3373, FOLFOX, FOLFOX4, FOLFIRI, MOF, deflexifol, or a combination of 5-FU with one or more selected from radiation, methyl-CCNU, leucovorin, oxaliplatin (such as cisplatin), irinotecan, mitomycin, cytarabine, and levamisole.

In some embodiments, the additional therapeutic agent comprises an inhibitor of folate-mediated one-carbon metabolism. In another aspect, the additional therapeutic agent comprises anthracycline or other topoisomerase II inhibitor comprises daunorubicin, doxorubicin, epirubicin, idarubicin, valrubicin, mitoxantrone, etoposide and teniposide.

In some embodiments, the additional therapeutic agent comprises one or more selected from monoclonal antibodies, optionally selected from a monospecific antibody, a bispecific antibody, multispecific antibody, a bispecific immune cell engager, or an antibody-drug conjugate. Non-limiting examples of monoclonal antibodies are selected from rituximab, blinatumomab, alemtuzumab, ibritumomab tiuxetan, bevacizumab, bevacizumab-awwb, cetuximab, panitumumab, ofatumumab, denosumab, pertuzumab, obinutuzumab, elotuzumab, ramucirumab, dinutuximab, daratumumab, trastuzumab, trastuzumab-dkst, nivolumab, pembrolizumab, cemiplimab, spartalizumab, camrelizumab, sintilimab, tislelizumab, toripalimab, AMF 514 (3EDJ0680), balstilimab, avelumab, durvalumab, atezolizumab, ipilimumab, tremelimumab, zalifrelimab, and AGEN1181. Non-limiting examples of antibody-drug conjugates are selected from moxetumomab pasudotox-tdfk, brentuximab vedotin, trastuzumab emtansine, inotuzumab ozogamicin, gemtuzumab ozogamicin, tagraxofusp-erzs, polatuzumab vedotin-piiq, enfortumab vedotin-ejfv, trastuzumab deruxtecan, and sacituzumab govitecan-hziy.

In some embodiments, the additional therapeutic agent comprises a CAR therapy selected from a CAR NK therapy, a CAR-T therapy, a CAR cytotoxic T therapy, or a CAR gamma-delta T therapy. In some embodiments, the CAR therapy is a CAR T-cell therapy selected from tisagenlecleucel and axicabtagene ciloleucel.

In some embodiments, the additional therapeutic agent comprises an immune regulator. In some embodiments, the immune regulator is selected from an interleukin, an aldesleukin, interferon alfa-2a/2b, pexidartinib, erythropoietin, granulocyte-macrophage colony-stimulating factor (GM-CSF), granulocyte colony-stimulating factor (G-CSF), thalidomide, lenalidomide, pomalidomide, or imiquimod.

In some embodiments, the additional therapeutic agent comprises a cancer vaccine. In some embodiments, the cancer vaccine is selected from CG live (THERACYS®) and sipuleucel-T (PROVENGE®).

In some embodiments, the additional therapeutic agent comprises a checkpoint inhibitor. In some embodiments, the checkpoint inhibitor is selected from GS4224, AMP-224, CA-327, CA-170, BMS-1001, BMS-1166, peptide-57, M7824, MGD013, CX-072, UNP-12, NP-12, or a combination of two or more thereof.

Additional checkpoint inhibitors comprises one or more selected from an anti-PD-1 agent, an anti-PD-L1 agent, an anti-CTLA-4 agent, an anti-LAG-3 agent, an anti-TIM-3 agent, an anti-TIGIT agent, an anti-VISTA agent, an anti-B7-H3 agent, an anti-BTLA agent, an anti-ICOS agent, an anti-GITR agent, an anti-4-1BB agent, an anti-OX40 agent, an anti-CD27 agent, an anti-CD28 agent, an anti-CD40 agent, and an anti-Siglec-15 agent. In a further aspect, the checkpoint inhibitor comprises an anti-PD1 agent or an anti-PD-L1 agent. In one aspect, the anti-PD1 agent comprises an anti-PD1 antibody or an antigen binding fragment thereof. In a further aspect, the anti-PD1 antibody comprises nivolumab, pembrolizumab, cemiplimab, spartalizumab, camrelizumab, sintilimab, tislelizumab, toripalimab, AMF 514, or a combination of two or more thereof. In another aspect, the anti-PD-L1 agent comprises an anti-PD-L1 antibody or an antigen binding fragment thereof. In a further aspect, the anti-PD-L1 antibody comprises avelumab, durvalumab, atezolizumab, envafolimab, or a combination of two or more thereof.

In some embodiments, the checkpoint inhibitor comprises an anti-CTLA-4 agent. In another aspect, the anti-CTLA-4 agent comprises an anti-CTLA-4 antibody or an antigen binding fragment thereof. In a yet further aspect, the anti-CTLA-4 antibody comprises ipilimumab, tremelimumab, zalifrelimab, or AGEN1181, or a combination thereof.

Another aspect of the disclosure is directed to an isolated host cell comprising the modified oHSV of the instant disclosure. In some embodiments, the isolated host cell produces modified oHSVs of the instant disclosure. In some embodiments, the cell is a prokaryotic or a eukaryotic cell. In some embodiments, the cell is a mammalian cell. In some embodiments, the cell is a human cell.

oHSV Vectors

The present disclosure also provides vectors (“oHSV vectors”) that can be used to produce an oncolytic herpes simplex virus (oHSV) that expresses a histidine-rich glycoprotein (HRG), optionally wherein the oHSV comprises an attenuated oHSV.

In some embodiments, the oHSV comprises an HSV-1 strain or an HSV-2 strain, optionally wherein the HSV-1 strain is selected from HSV-1 strain is selected from strain F (ATCC: VR-733), strain KOS (ATCC: VR-1493), strain HF (VR-260), strain McIntyre (VR-539), or any clinical isolate thereof; and optionally wherein the HSV-2 strain is selected from strain G (ATCC: VR-734), strain ATCC-2011-2 (ATCC: VR-1779), strain MS (ATCC: VR-540), ATCC-2011-4 (VR-1781), or any clinical isolate thereof.

In some embodiments, the HRG comprises an amino acid sequence with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: 1. In a further aspect, the HRG comprises, or consists essentially of, or consists of SEQ ID NO: 1.

In some embodiments, the HRG comprises an amino acid sequence with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: 2. In a further aspect, the HRG comprises, or consists essentially of, or consists of SEQ ID NO: 2.

In some embodiments, the HRG is fused to a SecA signal peptide. In some embodiments, the SecA signal peptide comprises, or consists essentially of, or consists of SEQ ID NO: 3 or an equivalent thereof with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: 3. In some embodiments, the SecA signal peptide is encoded by a nucleic acid that comprises, or consists essentially of, or consists of SEQ ID NO: 11 or an equivalent thereof with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: 11, that encodes SEQ ID NO: 3.

In some embodiments, the oHSV vector further comprises regulatory elements (e.g., a promoter, a tumor-specific promoter, or an enhancer) that regulate expression of HRG. In some embodiments, the oHSC vector comprises a CMV promoter and enhancer. In some embodiments, the CMV promoter and enhancer comprises a nucleic acid sequence that comprises, or consists essentially of, or consists of SEQ ID NO: 5 or an equivalent thereof with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: 5. In some embodiments, the oHSC vector comprises a Kozak sequence. In some embodiments, the Kozak sequence comprises, or consists essentially of or consists of SEQ ID NO: 10 or an equivalent thereof with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: 5. In some embodiments, the oHSC vector comprises a polyadenylation (polyA) signal sequence. In some embodiments, the polyA signal sequence comprises, or consists essentially of or consists of SEQ ID NO: 8 or an equivalent thereof with at least 80% (e.g., 80%, 81%, 82%, 83%, 84%, 85%, 86%, 87%, 88%, 89%, 90%, 91%, 92%, 93%, 94%, 95%, 96%, 97%, 98%, 99% or more) sequence identity to SEQ ID NO: 8.

In some embodiments, the oHSV further comprises a detectable or purification label, examples of such are known in the art and described herein.

In some embodiments, the oHSV vector has modifications, mutations, or deletion of at least one γ34.5 gene, which prevents oHSV replication in the central nervous system or dorsal root ganglia (see, Liu, B. L., et al., Gene therapy 10.4 (2003): 292-303). In some embodiments, the oHSV vector lacks intact γ34.5 genes. In some embodiments, both γ34.5 genes are deleted, mutated or modified. In other embodiments, one γ34.5 gene is deleted and the other γ34.5 gene is mutated or modified. In some embodiments, the terminal repeat, which comprises γ34.5 gene and ICP4 gene, is deleted.

In some embodiments, the oHSV vector comprises additional mutations, which may include disabling mutations (e.g., deletions, substitutions, insertions), which may affect the virulence of the virus or its ability to replicate. For example, mutations may be made in any one or more of ICP6, ICPO, ICP4, ICP27, ICP47, ICP 24, or ICP56. Preferably, a mutation in one of these genes (optionally in both copies of the gene where appropriate) leads to an inability (or reduction of the ability) of the HSV to express the corresponding functional polypeptide.

In some embodiments, the oHSV vector encodes an oHSV wherein both γ34.5 genes are deleted and the oHSV comprises an hHRG with a SecA signal sequence (the 17Δ34.5SecAhHRGvariant). In some embodiment, the 17Δ34.5SecAhHRG variant comprises, or consists essentially of, or consists of SEQ ID NO: 4. In some embodiments, the oHSV vector encodes an oHSV wherein the ICP6 are deleted and the oHSV comprises an hHRG with a SecA signal sequence (the Mut3ΔICP6SecAhHRG variant). In some embodiment, the Mut3ΔICP6SecAhHRG variant comprises, or consists essentially of, or consists of SEQ ID NO: 12.

In some embodiments, the oHSV vector may also have genes and nucleotide sequences that are non-HSV in origin. In some embodiments, the non-HSV sequences comprise a sequence that encodes a prodrug, a sequence that encodes a cytokine or other immune stimulating factor, a tumor-specific promoter, an inducible promoter, an enhancer, a sequence homologous to a host cell, among others may be in the oHSV genome. Exemplary non-HSV sequences include sequences encode IL12, IL15, OX40L, PD-L1 blocker or a PD-1 blocker. For sequences that encode a product, they are operatively linked to a promoter sequence and other regulatory sequences (e.g., enhancer, polyadenylation signal sequence) necessary or desirable for expression.

In some embodiments, the regulatory region of viral genes are modified to comprise response elements that affect expression. Exemplary response elements include response elements for N F-KB, Oct-3/4-SOX2, enhancers, silencers, cAMP response elements, CAAT enhancer binding sequences, and insulators. Other response elements may also be included. A viral promoter may be replaced with a different promoter. The choice of the promoter will depend upon a number of factors, such as the proposed use of the HSV vector, treatment of the patient, disease state or condition, and ease of applying an inducer (for an inducible promoter). For treatment of cancer, generally when a promoter is replaced, it will be with a cell-specific or tissue-specific or tumor-specific promoter. Tumor-specific, cell-specific and tissue-specific promoters are known in the art. Other gene elements may be modified as well. For example, the 5′ UTR of the viral gene may be replaced with an exogenous UTR.

Another aspect of the disclosure is directed to a population of modified oHSV prepared as described herein, wherein the modified oHSV are the same or different from each other. One of skill in the art can produce the population by admixing the modified oHSV in the ratios and selection of components as desired.

Another aspect of the disclosure is directed to a composition comprising the modified oHSVs prepared using the oHSV vector of the instant disclosure and/or the population modified oHSVs of the instant disclosure. In some embodiments, the composition further comprises a carrier, optionally a pharmaceutically acceptable carrier. In some embodiments, the composition further comprises an additional therapeutic agent, optionally an anticancer therapeutic agent. One of skill in the art can produce the composition by admixing the modified oHSV in the ratios and selection of components as desired in the carrier, such as a pharmaceutically acceptable carrier.

Another aspect of the instant disclosure is directed to an isolated host cell comprising the modified oHSV vector described herein. In some embodiments, the isolated host cell produces modified oHSVs of the instant disclosure. In some embodiments, the cell is a prokaryotic or a eukaryotic cell. In some embodiments, the cell is a mammalian cell. In some embodiments, the cell is a human cell.

### Methods for Inhibiting the Growth of a Cancer Cell

Also provided herein are in vitro and in vivo uses of the modified oHSV and oHSV vectors as described herein. They can be used in methods for inhibiting the growth of a cancer cell, the method comprising, or consisting essentially of, or yet further consisting of, contacting the cancer cell with an effective amount of the one or more of the modified oHSV, oHSV, or compositions as described herein under conditions to inhibit the growth of the cell. Contacting can be achieved by admixing the modified oHSV, oHSV, or compositions with the cell culture medium. The cell can be an animal cell, a mammalian cell, a rat or murine cell, a canine cell, a feline cell, a bovine cell, an equine cell, a simian cell or a human cell.

The cancer cell can be selected from a blood cancer cell, a sarcoma cell or a carcinoma cancer cell. Non-limiting examples include for example, a leukemic cell, a lymphoma cell, a colon cancer cell, a colorectal cancer cell, a rectal cancer cell, a gastric cancer cell, an esophageal cancer cell, a glioblastoma, a head and neck cancer cell, a breast cancer cell, a brain cancer cell, a lung cancer cell, a stomach cancer cell, a liver cancer cell, a gall bladder cancer cell, or a pancreatic cancer cell. In some embodiments, the cancer cell is a glioblastoma cancer cell.

One of skill in the art will appreciate that slowing of cancer cell growth or viability is an indication that the modified oHSV, oHSV, or compositions is or are successful. When the method is practiced in vitro, it provides an assay to test combination therapies or when the cancer cell is isolated from a subject's biopsy, to determine if the therapy is effective against the subject's cancer to provide a personalized therapy.

The method can also be practiced in vivo by administering to a subject in need thereof an effective amount of the one or more of the modified oHSV, oHSV, or compositions to the subject. One of skill in the art can determine if the therapy has been effective by noting a reduction in tumor burden, a reduction in metastasis, longer overall survival or longer progression free survival. The subject can be an animal, a mammal such as a pet or farm animal, or animal used for animal models, a rat or mouse, a canine, a feline, a bovine, an equine, a simian or a human.

Administration is systemic or localized. In some embodiments, the administration is intratumoral (itu) administration.

When the subject is an animal, such as a murine or rat, the method provides an animal model to test combination therapies or when the cancer cell is isolated from a subject's biopsy and grown in the animal model, it can be used to determine if the therapy is effective against the subject's cancer to provide a personalized therapy. When the subject is a pet, farm animal, or human, the method is a therapy to treat the pet, farm animal or human.

Thus, also provided herein is a method for treating cancer in a subject in need thereof, the method comprising, or consisting essentially of, or consisting of administering to the subject suffering from the cancer an effective amount of the modified oncolytic herpes simplex virus (oHSV) described herein or the oHSV vector described herein or a composition as described herein.

In some embodiments, administering to a subject suffering from a cancer an effective amount of the modified oncolytic herpes simplex virus (oHSV) described herein or the oHSV vector described herein or a composition as described herein results in increased CD8 T cells and enhanced CD8/Treg cell ratio. In some embodiments, administering to a subject suffering from a cancer an effective amount of the modified oncolytic herpes simplex virus (oHSV) described herein or the oHSV vector described herein or a composition as described herein results in increased M1/M2 macrophage ratio.

In some embodiments, the cancer is selected from a blood cancer, a sarcoma or a carcinoma, non-limiting examples of such include leukemia, lymphoma, colon cancer, colorectal cancer, rectal cancer, gastric cancer, esophageal cancer, head and neck cancer, breast cancer, brain cancer, glioblastoma, lung cancer, stomach cancer, liver cancer, gall bladder cancer, or pancreatic cancer. In some embodiments, the cancer is glioblastoma.

In some embodiments, the subject is a mammal. In some embodiments, the subject is a human.

Administration is systemic or localized. In some embodiments, the administration is intratumoral (itu) administration. In a further aspect, the cancer is glioblastoma and administration comprises, or consists essentially of, or consists of itu.

In some embodiments, the method is a first-line therapy, a second-line therapy, a third-line therapy, a fourth-line therapy, or a fifth-line therapy.

In some embodiments, the method further comprises, or consists essentially of, or consists of administering an additional therapeutic agent, optionally an anticancer therapeutic agent. Administration of the additional therapeutic agent is systemic or localized. In some embodiments, the administration is intratumoral (itu) administration. In a further aspect, the cancer is glioblastoma and administration of the additional therapeutic agent comprises, or consists essentially of, or consists of itu.

In some embodiments, the additional therapeutic agent is selected from the group of: 5-fluorouracil (5-FU), pemetrexed, raltitrexed, nolatrexed, plevitrexed, GS7904L, capecitabine, methotrexate, pralatrexate, CT-900, NUC-3373, FOLFOX, FOLFOX4, FOLFIRI, MOF, deflexifol, or a combination of 5-FU with one or more selected from radiation, methyl-CCNU, leucovorin, oxaliplatin (such as cisplatin), irinotecan, mitomycin, cytarabine, and levamisole.

In some embodiments, the additional therapeutic agent comprises an inhibitor of folate-mediated one-carbon metabolism. In another aspect, the additional therapeutic agent comprises anthracycline or other topoisomerase II inhibitor comprises daunorubicin, doxorubicin, epirubicin, idarubicin, valrubicin, mitoxantrone, etoposide and teniposide.

In some embodiments, the additional therapeutic agent comprises one or more selected from monoclonal antibodies, optionally selected from a monospecific antibody, a bispecific antibody, multispecific antibody, a bispecific immune cell engager, or an antibody-drug conjugate. Non-limiting examples of monoclonal antibodies are selected from rituximab, blinatumomab, alemtuzumab, ibritumomab tiuxetan, bevacizumab, bevacizumab-awwb, cetuximab, panitumumab, ofatumumab, denosumab, pertuzumab, obinutuzumab, elotuzumab, ramucirumab, dinutuximab, daratumumab, trastuzumab, trastuzumab-dkst, nivolumab, pembrolizumab, cemiplimab, spartalizumab, camrelizumab, sintilimab, tislelizumab, toripalimab, AMF 514 (3EDJ0680), balstilimab, avelumab, durvalumab, atezolizumab, ipilimumab, tremelimumab, zalifrelimab, and AGEN1181. Non-limiting examples of antibody-drug conjugates are selected from moxetumomab pasudotox-tdfk, brentuximab vedotin, trastuzumab emtansine, inotuzumab ozogamicin, gemtuzumab ozogamicin, tagraxofusp-erzs, polatuzumab vedotin-piiq, enfortumab vedotin-ejfv, trastuzumab deruxtecan, and sacituzumab govitecan-hziy.

In some embodiments, the additional therapeutic agent comprises a second CAR therapy selected from a CAR NK therapy, a CAR-T therapy, a CAR cytotoxic T therapy, or a CAR gamma-delta T therapy. In some embodiments, the second CAR therapy is a CAR T-cell therapy selected from tisagenlecleucel and axicabtagene ciloleucel.

In some embodiments, the additional therapeutic agent comprises an immune regulator. In some embodiments, the immune regulator is selected from an interleukin, an aldesleukin, interferon alfa-2a/2b, pexidartinib, erythropoietin, granulocyte-macrophage colony-stimulating factor (GM-CSF), granulocyte colony-stimulating factor (G-CSF), thalidomide, lenalidomide, pomalidomide, or imiquimod.

In some embodiments, the additional therapeutic agent comprises a cancer vaccine. In some embodiments, the cancer vaccine is selected from CG live (THERACYS®) and sipuleucel-T (PROVENGE®).

In some embodiments, the additional therapeutic agent comprises a checkpoint inhibitor. In some embodiments, the checkpoint inhibitor is selected from GS4224, AMP-224, CA-327, CA-170, BMS-1001, BMS-1166, peptide-57, M7824, MGD013, CX-072, UNP-12, NP-12, or a combination of two or more thereof.

Additional checkpoint inhibitors comprises one or more selected from an anti-PD-1 agent, an anti-PD-L1 agent, an anti-CTLA-4 agent, an anti-LAG-3 agent, an anti-TIM-3 agent, an anti-TIGIT agent, an anti-VISTA agent, an anti-B7-H3 agent, an anti-BTLA agent, an anti-ICOS agent, an anti-GITR agent, an anti-4-1BB agent, an anti-OX40 agent, an anti-CD27 agent, an anti-CD28 agent, an anti-CD40 agent, and an anti-Siglec-15 agent. In a further aspect, the checkpoint inhibitor comprises an anti-PD1 agent or an anti-PD-L1 agent. In one aspect, the anti-PD1 agent comprises an anti-PD1 antibody or an antigen binding fragment thereof. In a further aspect, the anti-PD1 antibody comprises nivolumab, pembrolizumab, cemiplimab, spartalizumab, camrelizumab, sintilimab, tislelizumab, toripalimab, AMF 514, or a combination of two or more thereof. In another aspect, the anti-PD-L1 agent comprises an anti-PD-L1 antibody or an antigen binding fragment thereof. In a further aspect, the anti-PD-L1 antibody comprises avelumab, durvalumab, atezolizumab, envafolimab, or a combination of two or more thereof.

In some embodiments, the checkpoint inhibitor comprises an anti-CTLA-4 agent. In another aspect, the anti-CTLA-4 agent comprises an anti-CTLA-4 antibody or an antigen binding fragment thereof. In a yet further aspect, the anti-CTLA-4 antibody comprises ipilimumab, tremelimumab, zalifrelimab, or AGEN1181, or a combination thereof.

Administration of the compounds within the combinations disclosed herein may be effected by any method that enables delivery of the compounds to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

In some embodiments, pembrolizumab is administered as a dose between about 100 mg and about 200 mg every 3 weeks. In some embodiments, nivolumab is administered as a dose between about 40 mg and about 240 mg once every 2 weeks. In some embodiments, nivolumab is administered as a dose between about 100 mg and about 480 mg once every 4 weeks. In some embodiments, ipilimumab is administered as a dose of about 1 mg/kg, 3 mg/kg, 5 mg/kg, 7 mg/kg or 10 mg/kg every 3 weeks for a total of 4 doses. In some embodiments, avelumab is administered as a dose between about 100 mg and about 800 mg every 2 weeks. In some embodiments, durvalumab is administered as a dose between 1 mg/kg and 10 mg/kg every 2 weeks. In some embodiments, atezolizumab is administered as a dose between 200 mg and 1200 mg intravenously over 60 minutes every 3 weeks.

In some embodiments, 5-FU is administered as a dose of 500 mg/m², i.v. bolus on day 1; and 1 hour prior to administering the 5-FU bolus, the patient is also administered leucovorin (500 mg/m², i.v.) over 2 hours. This regimen is repeated weekly on days 1, 8, 15, 22, 29, and 36 every 8 weeks for 4 to 6 cycles.

In some embodiments, 5-FU is administered in combination with radiation therapy. In further embodiments, 5-FU is administered as a dose of 500 mg/m², i.v. bolus for 5 days on days 1 and 36 beginning 22 to 70 days after surgery; and radiation therapy is administered for 6 weeks beginning on day 64 after initiation of 5-FU therapy, while 5-FU is administered at a dose of 225 mg/m²/day, i.v. continuous infusion throughout administration of radiation therapy. Then, 5-FU is administered at a dose of 450 mg/m², i.v. bolus daily for 5 days beginning 1 month after radiation (i.e., days 134 to 138) and repeated for 4 weeks.

In some embodiments, 5-FU is administered in combination with irinotecan and leucovorin, with or without bevacizumab (FOLFIRI with or without bevacizumab), wherein 5-FU is administered as a dose of 400 mg/m², i.v. bolus on day 1, followed by 5-FU 1,200 mg/m²/day on days 1 and 2 by continuous i.v. infusion (CIV) (total infusional dose, 2,400 mg/m²over 46 hours) for cycles 1 and 2. If there is no toxicity greater than grade 1, the 5-FU infusion dose may be increased to 3,000 mg/m²for all subsequent cycles.

In some embodiments, 5-FU is administered in combination with leucovorin and oxaliplatin with or without bevacizumab (FOLFOX4 with or without bevacizumab), wherein 5-FU is administered as a dose of 400 mg/m², i.v. bolus over 2 to 4 minutes, followed by 5-FU 600 mg/m² continuous i.v. infusion (CIV) over 22 hours on day 1. Prior to 5-FU bolus on day 1, oxaliplatin 85 mg/m², i.v. and leucovorin 200 mg/m², i.v. (both over 120 minutes via Y-site) are administered. If giving FOLFOX4 plus bevacizumab, bevacizumab 10 mg/kg, i.v. is administered over 30 to 90 minutes prior to chemotherapy on day 1. On day 2, a regimen of leucovorin 200 mg/m², i.v. over 2 hours followed by 5-FU 400 mg/m², i.v. bolus, followed by 5-FU 600 mg/m² CIV over 22 hours is repeated. The order of administration is bevacizumab followed by oxaliplatin and leucovorin, followed by 5-FU. This 2-day regimen is repeated every 2 weeks until disease progression or unacceptable toxicity is observed.

In some embodiments, capecitabine is administered as adjuvant following surgery (monotherapy) as a dose of 1.25 g/m² twice daily for 14 days, and subsequent courses are repeated after a 7-day interval, with recommended duration of treatment as 6 months, adjusted dose according to tolerability.

In some embodiments, capecitabine is administered as adjuvant following surgery (combination therapy) as a dose of 0.8-1 g/m² twice daily for 14 days, and subsequent courses are repeated after a 7-day interval, with recommended duration of treatment as 6 months, adjusted dose according to tolerability.

In some embodiments, capecitabine is administered as a dose of 1.25 g/m² twice daily for 14 days, and subsequent courses are repeated after a 7-day interval, adjusted dose according to tolerability.

In some embodiments, capecitabine is administered as a dose of 0.8-1 g/m² twice daily for 14 days, and subsequent courses are repeated after a 7-day interval, adjusted dose according to tolerability.

In some embodiments, capecitabine is administered in combination with a platinum based regimen, wherein capecitabine is administered as a dose of 0.8-1 g/m² twice daily for 14 days, and subsequent courses are repeated after a 7-day interval, or alternatively administered as a dose of 625 mg/m² twice daily given continuously, adjusted dose according to tolerability.

In some embodiments, methotrexate is administered orally or intramuscularly in doses of 15 to 30 mg daily for a five-day course. Such courses are usually repeated for 3 to 5 times as necessary.

In some embodiments, methotrexate is administered as a dose of 10 to 25 mg/day orally for 4 to 8 days.

In some embodiments, methotrexate is administered as a dose of 5 to 50 mg once weekly. Dose reduction or cessation is guided by patient response and hematologic monitoring.

In some embodiments, methotrexate is used in combination with other agents. In further embodiments, in addition to high-dose methotrexate with leucovorin rescue, these agents may include doxorubicin, cisplatin, and the combination of bleomycin, cyclophosphamide and dactinomycin (BCD). The starting dose for high-dose methotrexate treatment is 12 grams/m².

The therapies can be combined with additional therapies, such as surgical resection. Thus, the therapies can be first-line, second-line, third-line, fourth-line, fifth-line therapy.

The methods of this disclosure are administered to treat a subject having a cancer to the end of reduction in the size or number of tumors or cancer cells, a longer progression-free survival, or a longer overall survival, or a reduction in side effects or toxicity. The can be combined with diagnostic tests that establish if the subject is suitable or likely to benefit from the therapy. Such diagnostic tests are known in the art and briefly described above.

### Methods for Changing the Polarization of Macrophages

Another aspect of the disclosure is directed to a method for changing the polarization of macrophages from M2-like to M1-like in a subject comprising, or consisting essentially of, or consisting of administering to the subject one or more of the modified oncolytic herpes simplex virus (oHSV), the oHSV vector, or compositions as described herein.

In some embodiments, M1-like macrophages are MHCII+. In some embodiments, M2-like macrophages are CD206+. In some embodiments, the MHCII and/or CD206 status of a macrophage is determined by flow cytometry.

In some embodiments, M1-like macrophages comprise an upregulation of Cxc110 and/or Tnfa genes. In some embodiments, M1-like macrophages comprise an upregulation of Cd206, Ccl22, and/or Arg1 genes. In some embodiments, upregulation of Cxcl10, Tnfa, Cd206, Ccl22, and/or Arg1 genes is determined by quantitative RT-PCR.

In some embodiments, the subject is a mammal. In some embodiments, the subject is a human.

In some embodiments, administering to a subject one or more of the modified oncolytic herpes simplex virus (oHSV), the oHSV vector, or compositions as described herein results in changing the polarization of macrophages from M2-like to M1-like in the subject.

In some embodiments, the administration is systemic administration. In some embodiments, the administration is local administration. In some embodiments, the administration is intratumoral (itu) administration.

### Methods for Expressing HRG in a Cell

A method for expressing HRG comprising growing a cell comprising one or more of the oHSV, the population of oHSVs, the oHSV vector as described herein under conditions that favor expression of HRG. In some embodiments, the cell is a prokaryotic or eukaryotic cell. In some embodiments, the cell is a mammalian cell. In some embodiments, the cell is a human cell.

The disclosure now being generally described, it will be more readily understood by reference to the following example which is included merely for purposes of illustration of certain aspects and embodiments of the present disclosure, and are not intended to limit the disclosure.

### EXAMPLES

### Example 1: Expression of HRG in HSV-HRG Infected Cells

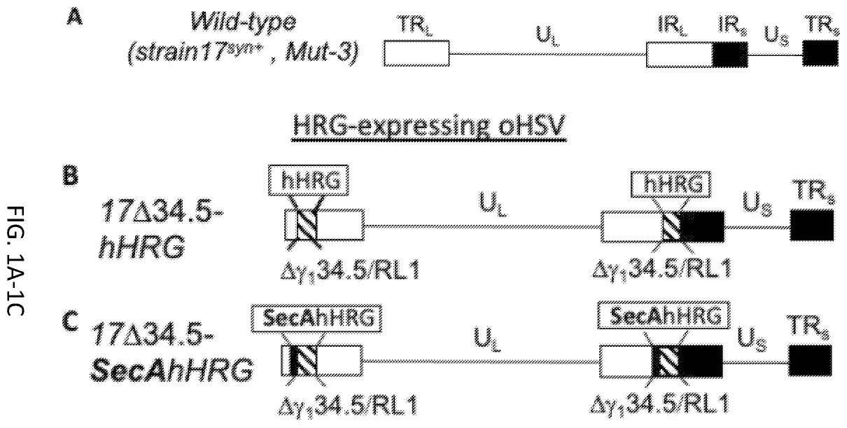

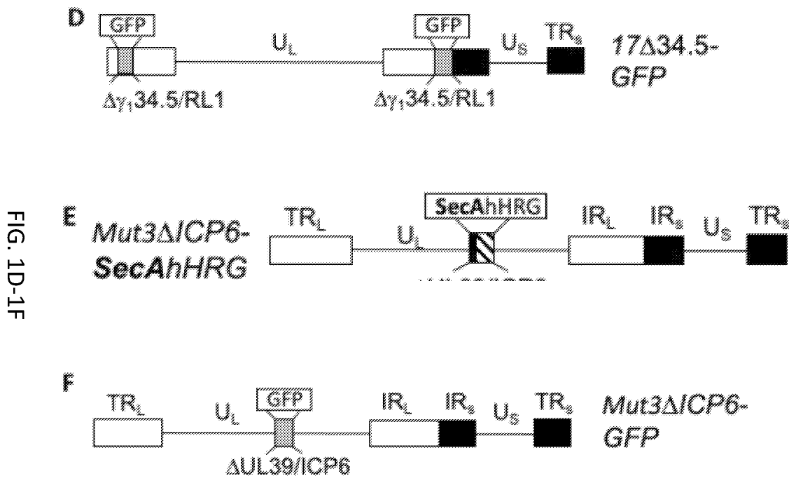

To clone the human histidine rich glycoprotein (hHRG)-expressing oncolytic HSV, both copies of γ₁34.5/RL1 (encodes ICP34.5) from wide-type HSV-1 strain 17^{syn+} were replaced with CMV-driven hHRG coding sequences, containing native signal peptide, as 17Δ34.5hHRG via CRISPR/Cas9 gene editing strategy. As the HRG protein secretion was barely detected from 17Δ34.5hHRG-infected cells, the native signal peptide sequence from hHRG was later replaced with a computationally-designed signal peptide, secrecon (Sec, peptide sequence MWWRLWWLLLLLLLLWPMVWA (SEQ ID NO: 3)) (Güler-Gane, G et Al., PLOS One 2016) with an additional alanine (A) at the end and clone into strain17^{syn+} as 17Δ34.5SecAhHRG. The control virus 17Δ34.5-GFP was generated via similar process by replacing γ₁34.5/RL1 with CMV-driven GFP expression cassette. To enhance virus lytic effect and increase viral replication to better target resistant tumors, Mut3ΔICP6SecAhHRG was generated by replacing UL39 (encodes ICP6, ribonucleotide reductase) with CMV-driven SecAhHRG expression cassette from a very potent wild-type like syncytia virus Mut-3. In parallel, control virus Mut3ΔICP6-GFP was developed with similar process by replacing UL39 with CMV-driven GFP expression cassette. See FIG. 1A-1F.

To validate HRG production, A673 cells were infected with 17Δ34.5-hHRG and hHRG secretion was measured using ELISA. After failing to detect much hHRG protein secretion in supernatant collected from 17Δ34.5-hHRG-infected A673 cells, Applicant replaced the native signal peptide of hHRG with an artificial signal peptide (17Δ34.5-SecAhHRG) and repeated the study (FIG. 1G). hHRG production was much higher in cells infected with 17Δ34.5-SecAhHRG compared to 17Δ34.5-hHRG, suggesting the SecA peptide greatly enhances hHRG secretion.

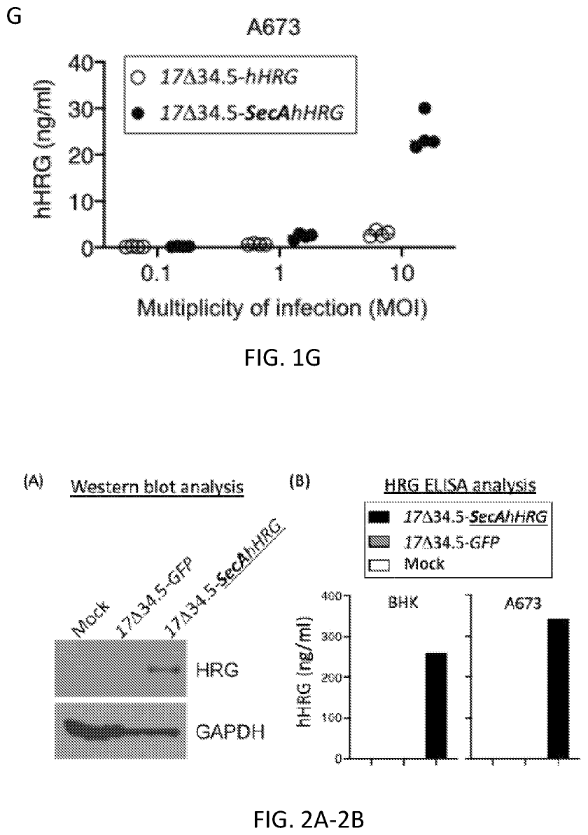

To confirm the size of the hHRG, baby hamster kidney (BHK) fibroblast cell line was infected with 17Δ34.5-SecAhHRG virus at the MOI=1 and cell lysates were prepared one day after infection for western blot analysis. Cell lysate was also prepared from 17Δ34.5-GFP-infected BHK cells as control. Full-length hHRG (molecular weight ˜70 kDa) was only detected in 17Δ34.5-SecAhHRG virus-infected cells (FIG. 2A). HRG protein secretion was quantified in the supernatants of infected BHK cells or the human Ewing sarcoma cell line A673 using ELISA reagents from R&D Systems and again hHRG protein secretions were only detected in cells infected with the 17Δ34.5-SecAhHRG virus (FIG. 2B).

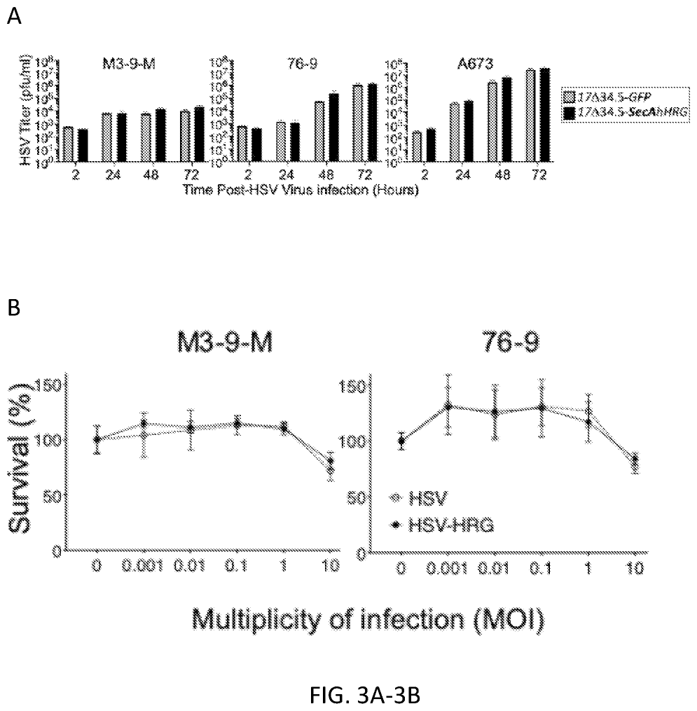

The permissivity of the two murine rhabdomyosarcoma (RMS) cell lines, M3-9-M and 76-9, to 17Δ34.5-SecAhHRG (HSV-HRG) and parental 17Δ34.5-GFP (HSV) virus in vitro was determined by assessing virus production (plague assay) over time following exposure to virus at low multiplicities of infection (MOI=0.01). Both viruses displayed similar replication kinetics (FIG. 3A).

In addition, sensitivities of the two murine cell lines to 17Δ34.5-SecAhHRG (HSV-HRG) and 17Δ34.5-GFP (HSV) were compared. Both viruses showed the same cytotoxicity in two mRMS cells (FIG. 3B). These data suggest that the oncolytic ability of HSV-HRG remains similar to parental HSV virus.

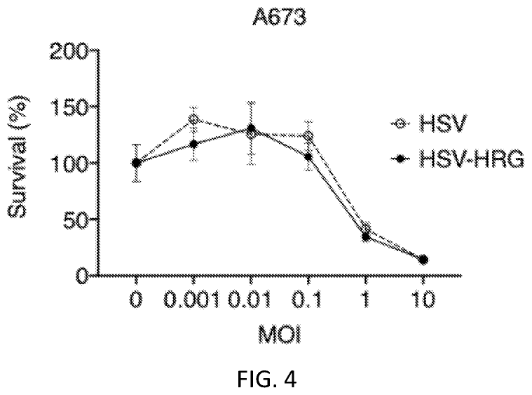

Applicant also compared the sensitivity of the A673 cell line (Ewing tumor) to HSV-HRG and HSV parental virus in MTS assays. Compared to murine RMS cell lines, both viruses were able to kill A673 cells at a lower MOI, suggesting A673 cell line was more sensitive to HSV treatment than murine RMS cell lines (FIG. 4). There is no difference between 17Δ34.5-SecAhHRG (HSV-HRG) and 17Δ34.5-GFP (HSV) viruses in this cytotoxicity assay, suggesting HRG did not affect oncolytic activity in A673 tumor cells.

### Example 2: HRG does not Alter HSV-Induced Chemokine and Cytokine Expressions In Vitro

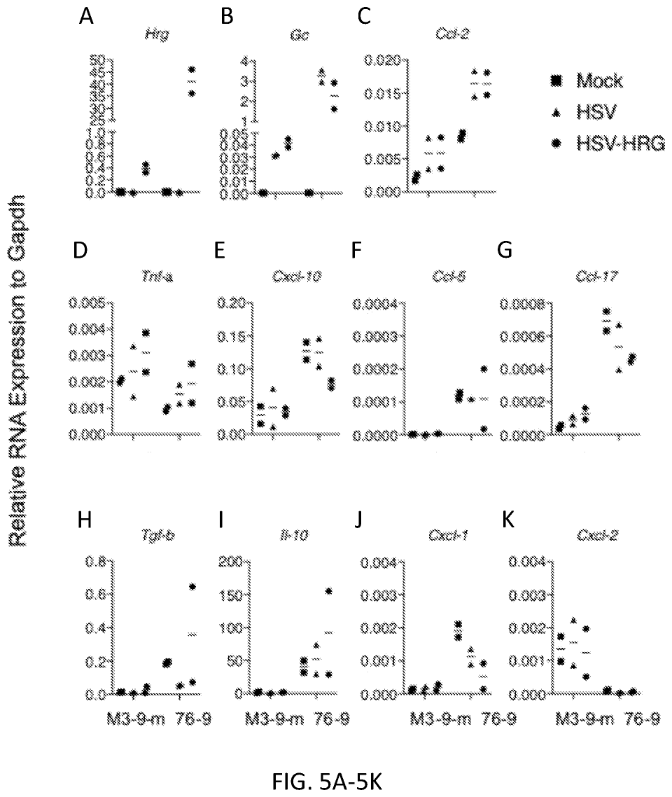

To explore if 17Δ34.5-SecAhHRG (HSV-HRG) treatment induces differential gene expression, two murine RMS cell lines were infected with 17Δ34.5-SecAhHRG (HSV-HRG) or 17Δ34.5-GFP (HSV) and compared their chemokine and cytokine gene expressions to mock-infected cells. The viral gene, gc, was highly induced in HSV- or HSV-HRG-infected tumor cell lines (FIG. 5). As expected, only cells infected with HSV-HRG expressed HRG transcripts. Expression of gc and HRG transcripts were much higher in 76-9 RMS cells than in M3-9-M RMS cells, suggesting 76-9 cells are more susceptible to HSV infection than M3-9-M cells (FIG. 5). Further comparative gene analysis showed that both viruses induce similar level of chemokine and cytokine expressions in the two RMS lines.

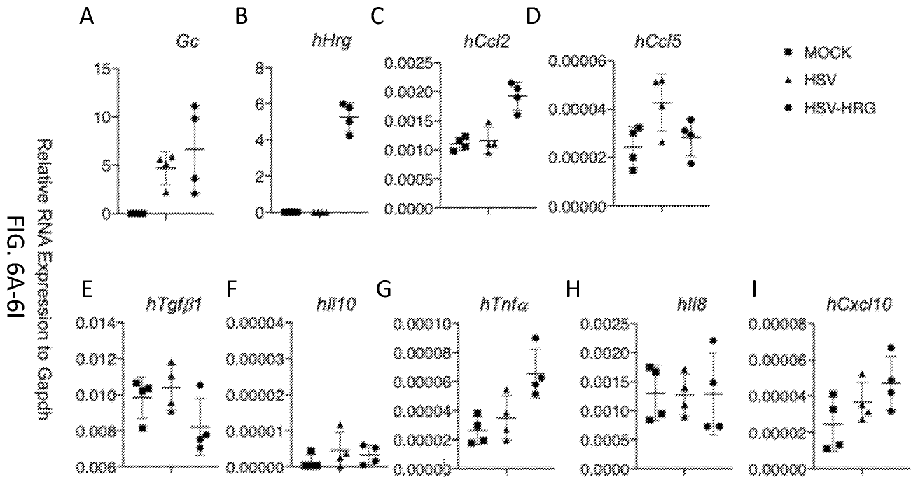

Applicant also compared gene expression profiles in A673 cell line treated with medium only, 17Δ34.5-GFP (HSV) and 17Δ34.5-SecAhHRG (HSV-HRG). Similar to what was observed in mRMS cell lines, viral transcript (gc) was detected in HSV- or HSV-HRG-infected A673 cells (FIG. 6). High levels of HRG transcript are only detected in HSV-HRG-infected cells and both viruses induce similar level of chemokine and cytokine expression in A673 cell line (FIG. 6).

### Example 3: HSV-HRG Induces M1 Macrophage Polarization and Reduces M2 Macrophage Polarization In Vitro and In Vivo

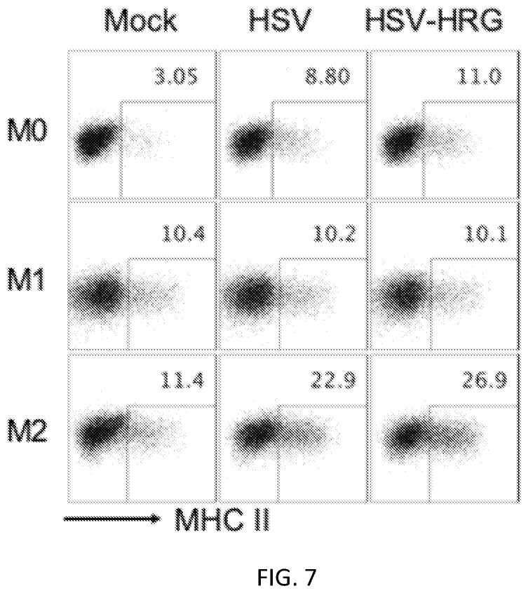

To determine the effect of 17Δ34.5-SecAhHRG (HSV-HRG) on M1 macrophage polarization, murine bone marrow cells were harvested and induced into M0, M1 and M2 macrophages using M-CSF, LPS and IL-4 respectively. M0, M1 or M2 macrophages were infected with 17Δ34.5-SecAhHRG (HSV-HRG), 17Δ34.5-GFP (HSV) or a mock control and evaluated M1 macrophage polarization by looking for the induction of MHC class II, a M1 macrophage marker, via flow cytometry (FIG. 7). It was found that HSV-HRG was able to induce more MHC II+M1-like macrophages from M0 or M2 macrophages compared to the other controls.

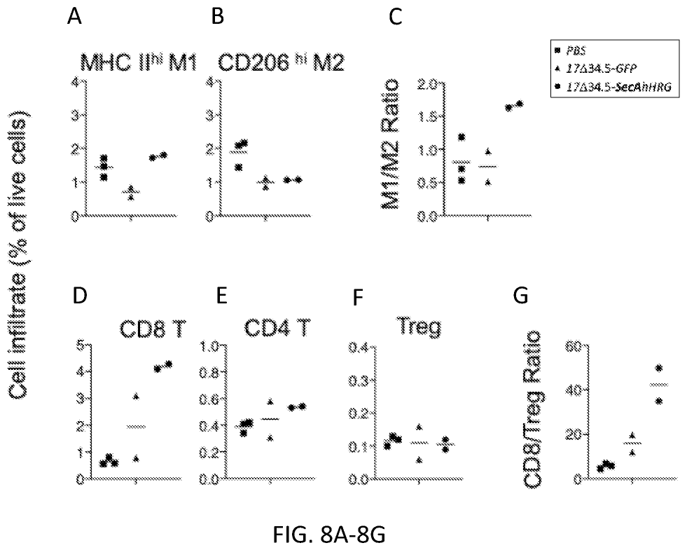

To investigate the effect of 17Δ34.5-SecAhHRG (HSV-HRG) on M1 macrophage polarization in vivo, male C57B/6 mice were subcutaneously implanted with 1×10⁶ M3-9-M RMS cells. Around 10 days later, the established tumors (˜250 mm³) were treated with PBS, 17Δ34.5-GFP (HSV) or 17Δ34.5-SecAhHRG (HSV-HRG) every other day for three doses. Three days after the last dose of virus treatment, tumor samples were harvested and single cell suspensions were prepared for flow cytometry analysis (FIG. 8). Tumor-bearing mice treated with HSV-HRG showed reduced levels of pro-tumorigenic CD206+M2-like macrophages without the concomitant drop in MHC II+M1-like macrophages observed in the parental HSV group, compared to PBS treatment. Compared to PBS control group, neither of the viruses treatments recruit more CD4 or Foxp3+ Treg cells. However, HSV-HRG treatment induced more CD8 T cells and enhanced CD8/Treg cell ratio, a positive prognostic factor in several type of cancers. It was also found that HSV-HRG treatment increased M1/M2 ratio, which potentially contributed to the greater influx of CD8+ T cells observed in this tumor model.

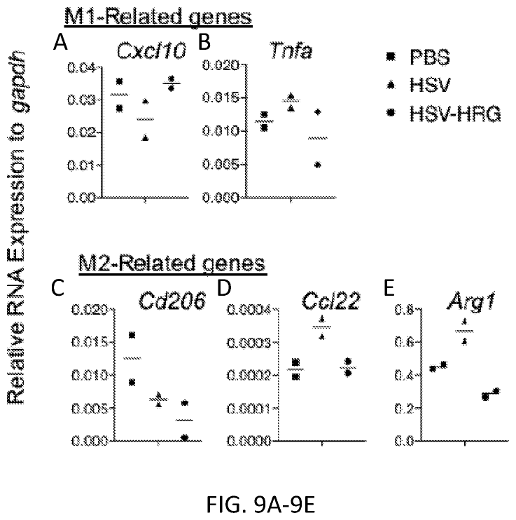

To evaluate the effects of 17Δ34.5-SecAhHRG (HSV-HRG) on macrophage polarization in vivo, M3-9-M tumors were harvested from mice treated with PBS, HSV and HSV-HRG. F4/80+ macrophages were enriched using a magnetic cell separation system from Miltenyi Biotec. M1 and M2 related gene expressions were then compared using quantitative real-time PCR. It was found that macrophages from 17Δ34.5-SecAhHRG (HSV-HRG) treated tumors express lower M2-related genes, CD206 and ARG1, compared to 17Δ34.5-GFP (HSV) or PBS treated groups (FIG. 9), suggesting HSV-HRG reduced M2 macrophage polarization in M3-9-M tumor model.

### Example 4: Expression of HRG Enhances oHSV Antitumor Efficacy in A673 Tumor Model

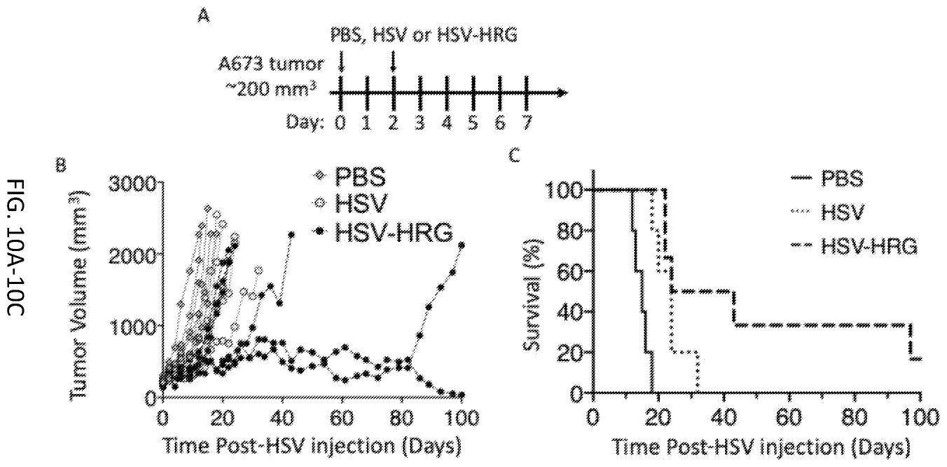

To explore if expression of HRG enhances oHSV virotherapy in A673 Ewing's tumors, A673 cells were implanted into nude mice and treated establish tumor (˜200 mm³) with 17Δ34.5-GFP (HSV), 17Δ34.5-SecAhHRG (HSV-HRG) or PBS followed by tumor measurement twice weekly. Tumor-bearing mice treated with 17Δ34.5-SecAhHRG (HSV-HRG) delayed tumor growth and prolonged survival compared to those received PBS or 17Δ34.5-GFP (HSV) treatment (FIGS. 10A-10C).

### Example 5: Mut3ΔICP6-SecAhHRG Further Enhances hHRG Secretion In Vitro and In Vivo

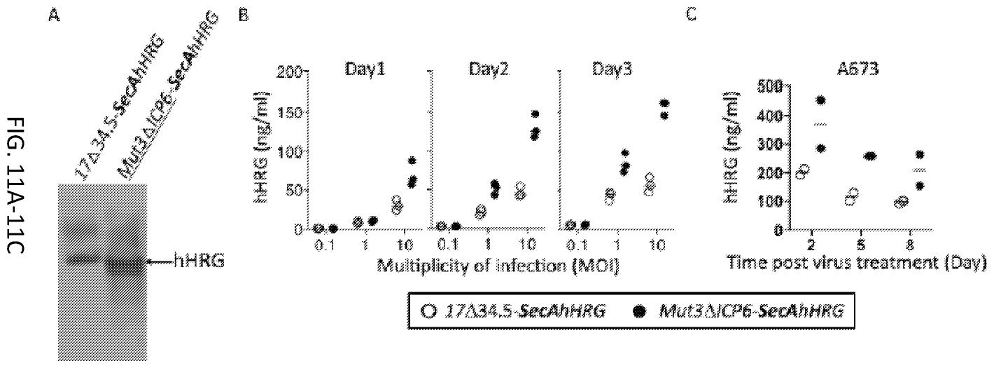

To be able to target tumors that are less sensitive to oHSV infection, a hHRG-expressing oHSV (Mut3ΔICP6-SecAhHRG) was created. hHRG protein expression was confirmed from this new construct using western blot analysis (FIG. 11A). Similarly, hHRG secretion was confirmed by ELISA, finding that Mut3ΔICP6-SecAhHRG infection further enhanced hHRG secretion in vitro and in vivo (FIGS. 11B and 11C).

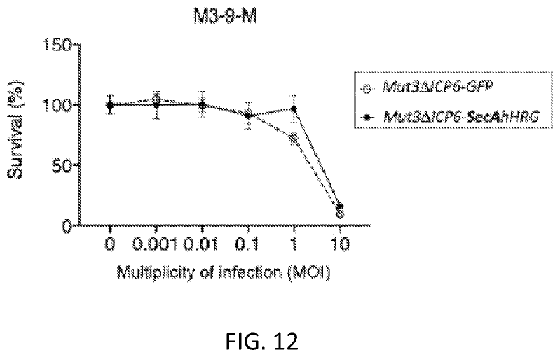

Applicant used viability assays to examine if arming the Mut3ΔICP6 virus with hHRG changed its tumor killing activity. Mut3ΔICP6-SecAhHRG virus did not change its tumor killing activity. Both Mut3ΔICP6-SecAhHRG and Mut3ΔICP6-GFP viruses displayed similar cell killing activity in M3-9-M tumor cell line (FIG. 12).

### Equivalents

Thus, it should be understood that although the present disclosure has been specifically disclosed by preferred embodiments and optional features, modification, improvement and variation of the disclosure embodied therein herein disclosed can be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. Several references are identified by an Arabic number, and the full bibliographic citation or these references are provided below. In case of conflict, the present specification, including definitions, will control.

1. A modified oncolytic herpes simplex virus (oHSV) comprising an expression cassette encoding a histidine-rich glycoprotein (HRG), wherein the oHSV comprises an attenuated oHSV.

2. The modified oHSV of claim 1, wherein the oHSV comprises HSV-1 strain or an HSV-2 strain, wherein the HSV-1 strain is selected from HSV-1 strain is selected from strain F (ATCC: VR-733), strain KOS (ATCC: VR-1493), strain HF (VR-260), strain McIntyre (VR-539), or any clinical isolate thereof, and wherein the HSV-2 strain is selected from strain G (ATCC: VR-734), strain ATCC-2011-2 (ATCC: VR-1779), strain MS (ATCC: VR-540), ATCC-2011-4 (VR-1781), or any clinical isolate thereof.

3. The modified oHSV of claim 1, wherein the HRG comprises an amino acid sequence with at least 80% sequence identity to SEQ ID NO: 1 or SEQ ID NO: 2.

4. The modified oHSV of claim 1, wherein the HRG is fused to a SecA signal peptide, wherein the SecA signal peptide comprises SEQ ID NO: 3 or the SecA signal sequence is encoded by SEQ ID NO: 11 or an equivalent of each thereof.

5. The modified oHSV of claim 1, wherein the oHSV comprises a gene encoding a dysfunctional ICP34.5 protein or a gene encoding a dysfunctional ICP6 protein.

6. The modified oHSV of claim 1, wherein oHSV comprises a gene encoding a dysfunctional ICP34.5 protein, a gene encoding human HRG, and a polynucleotide encoding SecA signal sequence located 5′ of the gene encoding human HRG; wherein the oHSV comprises SEQ ID NO: 9 or an equivalent thereof.

7. The modified oHSV of claim 1, wherein oHSV comprises a gene encoding a dysfunctional ICP6 protein, a gene encoding human HRG, and a polynucleotide encoding SecA signal sequence located 5′ of the gene encoding human HRG; wherein the oHSV comprises SEQ ID NO: 12 or an equivalent thereof.

8. The modified oHSV of claim 1, further comprising a Kozak sequence or a detectable or purification label, wherein the Kozak sequence comprises SEQ ID NO: 10 or an equivalent thereof.

9. (canceled)

10. A population or a composition of modified oncolytic herpes simplex viruses, comprising a plurality of modified oHSVs, wherein the oHSVs are selected from oHSVs of claim 1, wherein the modified oHSVs are the same or different from each other.

11-13. (canceled)

14. An isolated host cell comprising the modified oHSV of claim 1, wherein the cell is a prokaryotic or a eukaryotic cell.

15. (canceled)

16. An oncolytic herpes simplex virus (oHSV) vector encoding the modified oHSV of claim 1, wherein the oHSV comprises an HSV-1 strain or an HSV-2 strain, and wherein the HSV-1 strain is selected from HSV-1 strain is selected from strain F (ATCC: VR-733), strain KOS (ATCC: VR-1493), strain HF (VR-260), strain McIntyre (VR-539), or any clinical isolate thereof; and wherein the HSV-2 strain is selected from strain G (ATCC: VR-734), strain ATCC-2011-2 (ATCC: VR-1779), strain MS (ATCC: VR-540), ATCC-2011-4 (VR-1781), or any clinical isolate thereof.

17. (canceled)

18. The oHSV vector of claim 16, wherein the HRG comprises an amino acid sequence with at least 80% sequence identity to SEQ ID NO: 1 or SEQ ID NO: 2 or an equivalent of each thereof.

19. The oHSV vector of claim 16, further comprising a regulatory element that regulates expression of HRG; wherein the regulatory element is a promoter or an enhancer; wherein the regulatory element is a CMV promoter or a Kozak sequence, wherein the CMV promoter is as shown by SEQ ID NO: 5 or an equivalent thereof; and wherein the Kozak sequence comprises SEQ ID NO: 10 or an equivalent thereof.

20. The oHSV vector of claim 16, further comprising (i) polyadenylation signal located at the 3′ of an HRG sequence or (ii) a detectable or purification label.

21. (canceled)

22. A population of modified oHSV or a composition comprising a modified oHSV prepared using the oHSV vector of claim 16, wherein the modified oHSV are the same or different from each other.

23-25. (canceled)

26. An isolated host cell comprising the modified oHSV vector of claim 16, wherein the cell is a prokaryotic or a eukaryotic cell.

27. (canceled)

28. A method for expressing histidine-rich glycoprotein (HRG) comprising growing a cell comprising the modified oHSV of claim 1 under conditions that favor expression of HRG, wherein the cell is a prokaryotic or eukaryotic cell.

29. (canceled)

30. A method for inhibiting the growth of a cancer cell or treating cancer comprising administering to a subject suffering from the cancer an effective amount of the modified oncolytic herpes simplex virus (oHSV) of claim 1, wherein the cancer cell is selected from a cancer of the type: leukemia, lymphoma, colon cancer, colorectal cancer, rectal cancer, gastric cancer, esophageal cancer, head and neck cancer, breast cancer, glioblastoma, brain cancer, lung cancer, stomach cancer, liver cancer, gall bladder cancer, or pancreatic cancer.

31. (canceled)

32. The method of claim 30, wherein the cancer is glioblastoma, and wherein the subject is a mammal.

33-34. (canceled)

35. The method of claim 30, wherein the administration is intratumoral administration.

36. (canceled)

37. A method for changing the polarization of macrophages from M2-like to M1-like in a subject comprising administering to the subject the modified oncolytic herpes simplex virus (oHSV) of claim 1, wherein the administration is systemic or local administration.

38-40. (canceled)
